(19) United States
(12) Patent Application Publication
(10) Pub. No.: US 2019/0247376 A1
(43) Pub. Date: Aug. 15, 2019

(54) NICOTINIC ACETYLCHOLINE RECEPTOR AGONIST ATTENUATES ILC2-DEPENDENT AIRWAY HYPERREACTIVITY

(71) Applicant: University of Southern California, Los Angeles, CA (US)

(72) Inventors: Omid AKBARI, Los Angeles, CA (US); Lin CHEN, Los Angeles, CA (US)

(21) Appl. No.: 16/336,879

(22) PCT Filed: Sep. 29, 2017
(86) PCT No.: PCT/US2017/054612

§ 371 Date: Mar. 26, 2019

Related U.S. Application Data
(60) Provisional application No. 62/403,014, filed on Sep. 30, 2016.

(51) Int. Cl. A61K 31/444; A61K 45/06; A61P 29/00; G01N 33/50

(52) U.S. Cl. CPC A61K 31/444; A61K 45/06; G01N 2800/7095; G01N 33/5047; A61P 29/00

(57) ABSTRACT

This disclosure provides methods and compositions for treating diseases and disorders by targeting ILC2s that express the α7-nicotinic acetylcholine receptor (α7nAChR).

### CROSS-REFERENCE TO RELATED APPLICATION

This application claims the benefit under 35 U.S.C. 119(e) of U.S. Provisional Ser. No. 62/403,014, filed Sep. 30, 2016, the contents of which is hereby incorporated by reference into the present disclosure.

### STATEMENT OF FEDERAL SUPPORT

This invention was made with government support under the Grant No. TR000130, awarded by the National Institute for Health. Accordingly, the U.S. Government has certain rights to the invention.

### BACKGROUND

Throughout this disclosure, various patent and technical publications are identified by an identifying citation or an Arabic numeral, the full citations for which are found immediately preceding the claims. These citations and the publications referenced within the present specification are incorporated by reference into the present disclosure to more fully describe the state of the art to which this invention pertains.

Asthma, which is a major worldwide health problem, is a chronic inflammatory disease of the airways with several phenotypes, comprised of both allergic and non-allergic asthma^{1,2}. Allergic sensitization in which antigen presenting cells (APCs) present allergens, followed by T-helper type 2 (Th2) cell skewing and eosinophilic inflammation are essential for the development of allergic asthma. Obesity, ozone, viral infections, stress, and air pollution are associated with non-allergic asthma, the pathogenesis of which involves the innate pathway rather than Th2 cell mediated immunity³⁻⁵. Indeed, non-Th2 factors such as interferon-γ, IL-17, and neutrophils are often found in the lungs of patients with severe non-atopic asthma^{1,2}. Moreover, these allergic and non-allergic components may be present in individual patients to various degrees, leading to a complex immune milieu and disease heterogeneity^{1,2}.

Innate lymphoid cells (ILCs) are a non-B cell, non-T cell lymphocyte population in mucosal and lymphoid tissues that are not antigen specific, but respond rapidly to environment factors to induce various types of cytokines^{6,7}. Among the ILCs, group 2 ILCs (ILC2s) are directly activated by innate signals from myeloid and epithelial derived cytokines and alarmins, such as IL-25, IL-33 and proteases, without requiring further differentiation. Following activation, ILC2s produce robust amounts of Th2 cytokines IL-5 and IL-13 to promote eosinophilic inflammation and airway hyperreactivity (AHR); thus, they play an essential role in the pathogenesis of asthma^{6,7}. The suggestion that ILC2s are critical for innate immunity activation in asthma is logical as influenza infection⁵ and exposure to proteases and fungi^{8,9} induce AHR by activating innate lymphoid cells. In addition, ILC2s participate in shaping and regulating adaptive immune responses¹⁰. ILC2-produced IL-5 and IL-13 also contribute to asthma development by respectively recruiting eosinophils in airways and inducing goblet cell mucus production. ILC2s can also directly stimulate a Th2 response in vitro¹¹ and facilitate an antigen-specific T cell response during helminth infection¹². This evidence suggests that ILC2s are deeply involved in the pathogenesis of asthma by modulating both innate and adaptive immune responses. Thus, regulating the function of ILC2s could be an ideal therapeutic strategy.

### SUMMARY

The α7 nicotinic acetylcholine receptor (nAChR), mediates rapid excitatory synaptic transmission and is shown to be a potential therapeutic target in neuropsychiatric¹³, neurodegenerative¹⁴ and inflammatory disease^{15,16}. In pulmonary allergic inflammation, nicotine, an agonist for nAChR, attenuates Th2 cytokine, IgE, and cysteinyl leukotriene levels, resulting in reduced allergic inflammation^{17,18}. However, the specific cellular mechanisms by which nAChR activation regulates allergic inflammation are not clearly defined. Given the therapeutic potential of α7nAChR in neurological disorders and inflammatory diseases, a variety of α7-specific ligands have been developed by medicinal chemistry^{19,20} and structural approaches²¹⁻²³. A number of these α7nAChR compounds showed promising efficacy and have advanced to clinical trials¹⁹. Applicants selected a leading compound, GTS-21 (also known as DMXBA), that is functionally known to act as an agonist with partial selectivity toward α7nAChR^{24,25}, and characterize its role in ILC2-dependent AHR. Utilizing this agonist, Applicants demonstrate a critical role for α7nAChR in regulating ILC2-mediated AHR and airway inflammation in a preclinical model of allergic asthma. Moreover, Applicants validate the specificity of the α7nAChR agonist using α7nAChR deficient mice. As expected, the α7nAChR agonist was impaired in suppressing ILC2-dependent AHR in α7nAChR deficient mice. Reconstitution studies with alymphoid mice suggest that while recipients of wild-type ILC2s responded to suppressive activity of the agonist, recipients of the α7nAChR deficient ILC2s did not show any alteration in AHR or eosinophilia after agonist treatment. These observations establish that α7nAChR expression by ILC2s is crucial for the anti-inflammatory role of the agonist, and our reagent acts specifically through α7nAChR. Administration of the α7nAChR agonist inhibits activation of key transcriptional factors, such as GATA-3 and NF-κB. Furthermore, α7nAChR agonist abrogates phosphorylation of IKKα/β, a critical kinase upstream of the NF-κB signaling pathway. Using a translational approach, Applicants also show that engagement of α7nAChR results in decreased cytokine production in human ILC2s and a reduction in AHR in a humanized ILC2 model. These findings provide insight into the regulation of ILC2s that are useful to generate new therapeutic approaches for ILC2 dependent asthma.

Thus, as disclosed herein, methods are provided for one or more of:
a. reducing ILC2 effector function;
b. repressing IC2-dependent AHR;
c. decreasing expression of ILC2 transcription factor GATA-3;
d. decreasing expression of ILC2 inflammatory modulator NF-κB;
e. reducing phosphorylation of kinase IKKα/β; or
f. reducing ILC2-mediated cytokine production;
   in a tissue or subject in need thereof, the method comprising, or alternatively consisting essentially of, or yet further consists of, contacting an ILC2-cell expressing α7-nicotine acetylcholne receptor (α7nAChr) with an effective amount of an α7nAChr agonist. In one aspect, the an α7nAChr agonist comprises an active form of GTS-21, or an equivalent or a pharmaceutically acceptable salt thereof. In a further aspect, the cell is a mammalian cell such as for example, a human cell, a canine cell, a murine cell, a feline cell, a bovine cell or an equine cell.

When the methods are performed in vivo, an effective amount of the α7nAChr agonist is administered to the subject in need thereof. The methods are further modified by comprising administering an effective amount of an anti-inflammatory to the subject, that is optionally administered concurrently or sequentially in one or more administrations.

Also provided are methods for screening for an α7nAChr agonist, comprising, or alternatively consisting essentially of, or yet further consisting of, contacting an ILC2 cell expressing α7nAChr or tissue sample comprising ILC2 cells expressing α7nAChr, with a test agent and evaluating for the cell or tissue for one or more of:
a. reducing ILC2 effector function
b. decreasing expression of ILC2 transcription factor GATA-3;
c. decreasing expression of TLC2 inflammatory modulator NF-κB;
d. reducing phosphorylation of kinase IKKα/β;
e. reducing ILC2-mediated cytokine production;
f. treating and/or ameliorating AHR;
g. treating and/or ameliorating allergic inflammation; or
h. treating and/or ameliorating a disease related to ILC-2 effector function.
   wherein a measured response of a. to g. identifies the test agent as a possible α7nAChr agonist.

In a further aspect, the α7nAChr agonist such as GTS-21, or an equivalent or a pharmaceutically acceptable salt thereof is coadmininistered with an effective amount of an anti-inflammatory, such as a steroid or corticosteroid, or an antibiotic, an alpha or beta blocker, an antihistamine, a blocking antibody against Th2 cytokine and IgE. These can be administered concurrently or sequentially in one or more doses. In one aspect, an effective amount of the α7nAChr agonist such as GTS-21, or an equivalent or a pharmaceutically acceptable salt thereof is combined with an effective amount of the anti-inflammatory agent such as a steroid or corticosteroid, or antibiotics, alpha and beta blockers, antihistamine, blocking antibodies against Th2 cytokines and IgE, in a single formulation for simultaneous administration.

Further provided are methods for treating or ameliorating the symptoms of a disease or condition related to ILC2 effector function in a subject in need thereof, the method comprising administering an effective amount of an α7nAChr agonist to the subject, thereby treating or ameliorating the symptoms. In one aspect the α7nAChr agonist is GTS-21, or an equivalent or a pharmaceutically acceptable salt thereof. Non-limiting examples of a disease or condition related to ILC2 effector function include inflammatory disorders such as acute and chronic inflammatory disorders, COPD, IBS, Croh's disease, rheumatoid arthritis, allergic asthma, non-allergic asthma, lung inflammation, mucosal inflammation, nasal polyps, eczema, and atopic dermatitis.

In a further aspect, the α7nAChr agonist such as GTS-21, or an equivalent or a pharmaceutically acceptable salt thereof is coadmininistered with an effective amount of an anti-inflammatory, such as a steroid or corticosteroid, or an antibiotic, an alpha or beta blocker, an antihistamine, a blocking antibody against Th2 cytokine and IgE. These can be administered concurrently or sequentially in one or more doses. In one aspect, an effective amount of the α7nAChr agonist such as GTS-21, or an equivalent or a pharmaceutically acceptable salt thereof is combined with an effective amount of the anti-inflammatory agent such as a steroid or corticosteroid, or antibiotics, alpha and beta blockers, antihistamine, blocking antibodies against Th2 cytokines and IgE, in a single formulation for simultaneous administration.

### BRIEF DESCRIPTION OF THE DRAWING

FIGS. 1A-1E: Structure of α7nAChR agonist and α7nAChR expression on ILC2s. (FIG. 1A) Structure of 4-OH-GTS-21 bound to the α7nAChR Chimera. The receptor protein is shown in ribbon; the ligand and nearby protein residues are shown in stick model. The ligand, protein residues and key receptor loop (Loop C and Loop F) are labeled accordingly. (FIG. 1B) Flow cytometry analysis of lung ILC2s isolated from BALB/cByJ mice, as defined by a lack of lineage markers (CD3e, CD45R, Gr-1, CD11c, CD11b, Ter119, NK1.1, TCR-γδ and FcεRI) and expression of CD90, CD45 and ST2. Dot plots show cells gated on CD45+ and CD25+ single cells. (FIG. 1C) Peripheral immune cells were sorted from naïve BALB/cByJ mice and cultured with rm-IL-2 and rm-IL-7 in the presence or absence of rm-IL-33. The expressions of α7nAChR and α4nAChR were quantified at the mRNA levels by quantitative real-time PCR. (FIG. 1D) α7nAChR fold-change expression after IL-33 treatment in peripheral immune cells. (FIG. 1E) Expression of α7nAChR in isolated lung ILC2s from mice intranasally challenged for 3 consecutive days with PBS, IL-25 (5 μg), or IL-33 (0.5 μg). Data are representative of at least three independent experiments and are presented as means±SEM (n=3; Student's t test *, p<0.05).

FIGS. 2A-2C: α7nAChR agonist suppresses IL-5 and IL-13 production from ILC2s in vitro. BALB/cByJ mice were intranasally challenged with recombinant mouse IL-33 on days 1-3. On day 4 lung ILC2s were isolated and re-stimulated with rIL-33, rIL-2 and rIL-7 with or without α7nAChR agonist (2.5, 10 and 50 μg mL⁻¹) for 24 hours (FIG. 2A) or 48 hours (FIG. 2B). The levels of IL-5 and IL-13 were measured by ELISA. The viability of cultured ILC2s were assessed by staining with 7-AAD. (FIG. 2C) BALB/cByJ mice were intranasally challenged with recombinant mouse IL-33 on days 1-3, with or without α7nAChR agonist administration. On day 4 lung ILC2s were isolated and the number of IL-5⁺ and IL-13⁺ cells per 40×10⁴ ILC2s were quantified. Values are expressed as the mean±SEM of five experiments (n=5; Student's t test *, p<0.05; **p<0.01).

FIGS. 3A-3H: α7nAChR agonist treatment ameliorates ILC2s mediated AHR. (FIG. 3A) Rag2-deficient mice were intranasally challenged with recombinant mouse IL-33 or PBS and also received α7nAChR agonist (125 μg) or PBS on days 1-3 according to scheme 3A. Measurement of lung function and analyses of bronchoalveolar lavage fluid (BALF) and lung histology followed on day 4. (FIGS. 3B-3C) Lung resistance and dynamic compliance. (FIG. 3D) Total number of cells and eosinophils in BALF. (FIG. 3E) Hematoxylin and eosin-stained lung sections (×200). Scale bars represent 50 μm. (FIG. 3F) Quantification of lung histopathology shown as number of inflammatory cells per 250 μm² and thickness of airway epithelium (μm). (FIG. 3G) Total number and frequency of lung ILC2s. (FIG. 3H) Percentage of IL-5⁺ and IL-13⁺ lung ILC2s determined by flow cytometry. Data are representative of at least four independent experiments and are presented as means±SEM (n=8; Student's test *, p<0.05; **, p<0.01; ***, p<0.001).

FIGS. 4A-4E: α7nAChR expression is critical for GTS-21 action on ILC2s in vivo. BALB/cByJ and α7nAChR^{-/-} mice were intranasally challenged with rm-IL-33 or PBS, and also given α7nAChR agonist (125 μg) or PBS on days 1-3. Measurement of lung function and analysis of BAL followed on day 4. (FIG. 4A) Lung resistance. (FIG. 4B) Total number of eosinophils in BALF. (FIG. 4C) Rag^{-/-} GC^{-/-} mice were received ILC2s from either WT or α7nAChR^{-/-} mice. After the adoptive transfer, mice were intranasally challenged with rm-IL-33 or PBS; they also received α7nAChR agonist (125 μg) or PBS on days 1-3. Measurement of lung function and analysis of BAL followed on day 4, according to scheme 4C. (FIG. 4D) Lung resistance. (FIG. 4E) Total number of eosinophils in BALF. Data are representative of at least three independent experiments and are presented as means±SEM (n=4; Student's test ***, p<0.001, WT IL-33+α7nAChR agonist vs. α7nAChR^{-/-} IL-33+α7nAChR agonist).

FIGS. 5A-5F: Cholinergic signal attenuates ILC2 proliferation and GATA3 expression. (FIG. 5A) Mean fluorescence intensity of Ki67 and percentage of Ki67+ cells in isolated lung ILC2s, stimulated with IL-33 in the absence and presence of α7nAChR. (FIG. 5B) Mean fluorescence intensity of GATA3 in isolated lung ILC2s, stimulated with IL-33 in the absence and presence of α7nAChR. One representative experiment of two is shown (n=5; Student's t test *, p<0.05; **, p<0.01; ***p<0.001). (FIG. 5C) Heat plot demonstration of modulation of depicted genes in lung ILC2s treated with α7nAChR agonist. FACS-purified ILC2s from WT mice challenged with rm-IL-33 with or without α7nAChR agonist as described in FIG. 3A were quantified by NanoString nCounter technology. Data range from −1 to +1 for the most reduced and most increased gene expression, respectively. (FIG. 5D) Expression of NF-κB p65 in isolated lung ILC2s from mice challenged with IL-33 with (dotted line) or without (thick line) α7nAChR agonist. The level of isotype-matched stain control is shown as a gray-filled histogram. (FIG. 5E) Expression of phosphorylated IKKα/β (Ser176/180) in isolated lung ILC2s stimulated with IL-33, with (dotted line) or without (thick line) α7nAChR agonist for 24 hours. The level of isotype-matched stain control is shown as a gray-filled histogram. (FIG. 5F) Mean fluorescence intensity of phosphorylated IKKα/β with or without α7nAChR agonist.

FIGS. 6A-6C: α7nAChR agonist inhibits Alternaria-induced AHR. (FIG. 6A) Rag2-deficient mice intranasally received an extract of Alternaria alternata with or without α7nAChR agonist on days 1-4 according to scheme FIG. 6A. Measurement of lung function and BALF analysis followed on day 5. (FIG. 6B) Lung resistance. (FIG. 6C) Total number of eosinophils in BALF. Total number of lung ILC2s. Data are representative of at least four independent experiments and are presented as means±SEM (n=4; Student's t test *, p<0.05; ***, p<0.001).

FIGS. 7A-7E: α7nAChR agonist prevents human ILC2s mediated AHR and allergic. (FIG. 7A) Human ILC2s were purified from PBMCs via FACS and cultured (10⁴ cells per mL) in the presence of recombinant human IL-33 (20 ng mL⁻¹), IL-2 (10 ng mL⁻¹), and IL-7 (20 ng mL⁻¹), with or without α7nAChR agonist for 24h. In the right panel, data are presented as means of four individual donors. (FIG. 7B) Human peripheral-blood mononuclear cells were isolated via FACS and cultured in the presence or absence of rh-IL-2 (10 ng mL⁻¹) and rh-IL-7 (20 ng mL⁻¹) for 48 hours, then adoptively transferred into Rag2^{-/-} GC^{-/-} mice that were intranasally challenged with rm-IL-33 or PBS, with or without α7nAChR agonist (125 μg) on days 1-3. Measurement of lung function and analysis of BALF followed on day 4. (FIG. 7C) Lung resistance. (FIG. 7D) Total number of eosinophils in BALF. (FIG. 7E) Total number of lung ILC2s. Data are representative of at least two independent experiments and are presented as means±SEM (n=4; Student's test *, p<0.05; **, p<0.01; ***, p<0.001).

FIG. 8: Detection of α7nAChR in ILC2s by α-bungarotoxin. Histogram (left panel) and mean fluorescence intensity (right panel) of α7nAChR expression in ILC2s from BALB/cByJ mice after i.n. IL-33 (thick line) or PBS (dotted line). The level isotype-matched stain control is shown as a gray-filled histogram (left panel). Data are representative of at least two independent experiments and are presented as means±SEM (n=3; Student's t test *, p<0.05).

FIGS. 9A-9C: Effect of α7nAChR agonist on some marker expressions by ILC2s. A cohort of BALB/cByJ mice were intranasally challenged with rm-IL-33 (0.5 μg) with or without α7nAChR agonist (125 μg) for three consecutive days. Histogram and MFI of CD25 (FIG. 9A), CD127 (FIG. 9B) and ST2 (FIG. 9C) in isolated lung ILC2s. Data are representative of at least two independent experiments and are presented as means±SEM (n=4; Student's t test *, p<0.05).

FIG. 10: α7nAChR agonist suppresses cytokine production in ILC2s. Lung ILC2s were sorted from naïve BALB/cByJ mice and cultured with rm-IL-25 (10 ng mL−1) in the presence or absence of α7nAChR (10 μg mL−1) agonist for 24 hours. The levels of IL-5 and IL-13 were measured by ELISA. Data are representative of at least two independent experiments and are presented as means±SEM (n=4; Student's t test *, p<0.05).

FIGS. 11A-11C show the effect of nicotine agonists on ILCs in vitro. FIGS. 11A-11-C show the effect of TC-5619-23, TC-6987-23, AZD1446 on ILC2s.

FIGS. 12A-12C show the effect of nicotine agonists on ILCs in vitro. FIGS. 12A-12-C show the effect of PHA543613, PHA568487, PNU282987 on ILC2s.

### DETAILED DESCRIPTION

Before the compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; and Mayer and Walker eds. (1987) Immunochemical Methods in Cell and Molecular Biology (Academic Press, London).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above.

### Definitions

As used in the specification and claims, the singular form “a”, “an” and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated” as used herein with respect to nucleic acids, such as DNA or RNA, refers to molecules separated from other DNAs or RNAs, respectively that are present in the natural source of the macromolecule. The term “isolated peptide fragment” is meant to include peptide fragments which are not naturally occurring as fragments and would not be found in the natural state. The term “isolated” is also used herein to refer to polypeptides, antibodies, proteins, host cells and polynucleotides that are isolated from other cellular proteins or tissues and is meant to encompass both purified and recombinant polypeptides, antibodies, proteins and polynucleotides. In other embodiments, the term “isolated” means separated from constituents, cellular and otherwise, in which the cell, tissue, polynucleotide, peptide, polypeptide, protein, antibody or fragment(s) thereof, which are normally associated in nature and can include at least 80%, or alternatively at least 85%, or alternatively at least 90%, or alternatively at least 95%, or alternatively at least 98%, purified from a cell or cellular extract. For example, an isolated polynucleotide is separated from the 3′ and 5′ contiguous nucleotides with which it is normally associated in its native or natural environment, e.g., on the chromosome. An isolated cell, for example, is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

The term “binding” or “binds” as used herein are meant to include interactions between molecules that may be detected using, for example, a hybridization assay. The terms are also meant to include “binding” interactions between molecules. Interactions may be, for example, protein-protein, antibody-protein, protein-nucleic acid, protein-small molecule or small molecule-nucleic acid in nature. This binding can result in the formation of a “complex” comprising the interacting molecules. A “complex” refers to the binding of two or more molecules held together by covalent or non-covalent bonds, interactions or forces.

Hybridization reactions can be performed under conditions of different “stringency”. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

The term “polypeptide” is used interchangeably with the term “protein” and in its broadest sense refers to a compound of two or more subunit amino acids, amino acid analogs or peptidomimetics. The subunits may be linked by peptide bonds. In another embodiment, the subunit may be linked by other bonds, e.g., ester, ether, etc. As used herein the term “amino acid” refers to natural and/or unnatural or synthetic amino acids, including glycine and both the D and L optical isomers, amino acid analogs and peptidomimetics. A peptide of three or more amino acids is commonly called an oligopeptide if the peptide chain is short. If the peptide chain is long, the peptide is commonly called a polypeptide or a protein. The term “peptide fragment” as used herein, also refers to a peptide chain.

The phrase “equivalent polypeptide” or “biologically equivalent peptide or peptide fragment” or “biologically equivalent polynucleotide” refers to a protein or a peptide fragment which is homologous to the exemplified reference polynucleotide, protein or peptide fragment and which exhibit similar biological activity in vitro or in vivo, e.g., approximately 100%, or alternatively, over 90% or alternatively over 85% or alternatively over 70%, as compared to the standard or control biological activity. Additional embodiments within the scope of this invention are identified by having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence identity or homology. Percentage homology can be determined by sequence comparison using programs such as BLAST run under appropriate conditions. In one aspect, the program is run under default parameters.

The term “polynucleotide” refers to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, or EST), exons, introns, messenger RNA (mRNA), transfer RNA, ribosomal RNA, ribozymes, cDNA, RNAi, siRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

“Homology” or “identity” or “similarity” are synonymously and refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/blast/Blast.cgi, last accessed on Nov. 26, 2007. Biologically equivalent polynucleotides are those having the specified percent homology and encoding a polypeptide having the same or similar biological activity.

The term “non-contiguous” refers to the presence of an intervening peptide, nucleotide, polypeptide or polynucleotide between a specified region and/or sequence. For example, two polypeptide sequences are non-contiguous because the two sequences are separated by a polypeptide sequences that is not homologous to either of the two sequences. Non-limiting intervening sequences are comprised of at least a single amino acid or nucleotide.

A “gene” refers to a polynucleotide containing at least one open reading frame (ORF) that is capable of encoding a particular polypeptide or protein after being transcribed and translated. Any of the polynucleotide or polypeptide sequences described herein may be used to identify larger fragments or full-length coding sequences of the gene with which they are associated. Methods of isolating larger fragment sequences are known to those of skill in the art.

The term “express” refers to the production of a gene product such as RNA or a polypeptide or protein.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced there from.

Applicants have provided herein the polypeptide and/or polynucleotide sequences for use in gene and protein transfer and expression techniques described below. It should be understood, although not always explicitly stated that the sequences provided herein can be used to provide the expression product as well as substantially identical sequences that produce a protein that has the same biological properties. These “biologically equivalent” or “biologically active” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge.

A polynucleotide of this invention can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “composition” is intended to mean a combination of active polypeptide, polynucleotide or antibody and another compound or composition, inert (e.g. a detectable label) or active (e.g. a gene delivery vehicle) alone or in combination with a carrier which can in one embodiment be a simple carrier like saline or pharmaceutically acceptable or a solid support as defined below.

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject,” “individual” or “patient” is used interchangeably herein, and refers to a vertebrate, preferably a mammal, more preferably a human. Mammals include, but are not limited to, murines, rats, rabbits, simians, bovines, ovines, porcines, canines, felines, farm animals, sport animals, pets, equines, and primates, particularly humans.

“Cell,” “host cell” or “recombinant host cell” are terms used interchangeably herein. It is understood that such terms refer not only to the particular subject cell but to the progeny or potential progeny of such a cell. The cells can be of any one or more of the type murine, rat, rabbit, simian, bovine, ovine, porcine, canine, feline, equine, and primate, particularly human. Because certain modifications may occur in succeeding generations due to either mutation or environmental influences, such progeny may not, in fact, be identical to the parent cell, but are still included within the scope of the term as used herein.

As used herein, the term “ILC2 cell” an cell that is an innate immune cells cells are derived from common lymphoid progenitor (CLP) and belong to the lymphoid lineage. These cells are defined by absence of antigen specific B or T cell receptor because of the lack of recombination activating gene (RAG). ILCs do not express myeloid or dendritic cell markers. Group 2 ILCs (ILC2s) can produce type 2 cytokines (e.g. IL-4, IL-5, IL-9, IL-13). The cells (also termed natural helper cells, nuocytes, or innate helper 2 cells) have been implicated in the development of allergic lung inflammation, They express characteristic surface markers and receptors for chemokines, which are involved in distribution of lymphoid cells to specific organ sites. They require IL-7 for their development, which activates two transcriptional factors (both required by these cells)—ROR alpha and GATA3. After stimulation with Th2 polarising cytokines (e.g. IL-25, IL-33, TSLP) ILC2s start to produce IL-5, IL-13, IL-9, IL-4. ILC2s are critical for primary responses to local Th2 antigens e.g. allergens, helmints and viruses and that is why ILC2s are abundant in tissues of skin, lungs, livers and gut. Murine ILC2s can be (CD3e, CD45R, Gr-1, CD11c, CD11b, Ter119, NK1.1, TCR-ab and FCgammaRI) negative CD25+ CD90.2+ CD45+ and ST2+. Human ILC2s can be (CD3, CD14, CD16, CD19, CD20, CD56, CD235a, CD1a, CD123) negative cells, CRTH2⁺, CD161⁺, and CD45^{+.}

“Treating,” “treatment,” or “ameliorating” of a disease includes: (1) preventing the disease, i.e., causing the clinical symptoms of the disease not to develop in a patient that may be predisposed to the disease but does not yet experience or display symptoms of the disease; and/or (2) inhibiting the disease, i.e., arresting or reducing the development of the disease or its clinical symptoms; and/or (3) relieving the disease, i.e., causing regression of the disease or its clinical symptoms.

An effective amount of therapeutic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, and topical application.

The term “suffering” as it related to the term “treatment” refers to a patient or individual who has been diagnosed with or is predisposed to a disease. A patient may also be referred to being “at risk of suffering” from a disease. This patient has not yet developed characteristic disease pathology, however are known to be predisposed to the disease due to family history, being genetically predispose to developing the disease, or diagnosed with a disease or disorder that predisposes them to developing the disease to be treated.

The terms “inflammatory response” and “inflammation” as used herein indicate the complex biological response of vascular tissues of an individual to harmful stimuli, such as pathogens, damaged cells, or irritants, and includes secretion of cytokines and, more particularly, of pro-inflammatory cytokines, i.e. cytokines which are produced predominantly by activated immune cells and are involved in the amplification of inflammatory reactions. Exemplary pro-inflammatory cytokines include but are not limited to IL-1, IL-6, IL-10, TNF-α, IL-17, IL21, IL23, IL27 and TGF-β. Exemplary inflammations include acute inflammation and chronic inflammation. Acute inflammation indicates a short-term process characterized by the classic signs of inflammation (swelling, redness, pain, heat, and loss of function) due to the infiltration of the tissues by plasma and leukocytes. An acute inflammation typically occurs as long as the injurious stimulus is present and ceases once the stimulus has been removed, broken down, or walled off by scarring (fibrosis). Chronic inflammation indicates a condition characterized by concurrent active inflammation, tissue destruction, and attempts at repair. Chronic inflammation is not characterized by the classic signs of acute inflammation listed above. Instead, chronically inflamed tissue is characterized by the infiltration of mononuclear immune cells (monocytes, macrophages, lymphocytes, and plasma cells), tissue destruction, and attempts at healing, which include angiogenesis and fibrosis. An inflammation can be inhibited in the sense of the present disclosure by affecting and in particular inhibiting any one of the events that form the complex biological response associated with an inflammation in an individual. An anti-inflammatory is an agent or drug that provides this therapeutic response.

Examples of inflammatory disorders include, but are not limited to arthritis (rheumatoid arthritis such as acute arthritis, chronic rheumatoid arthritis, type II collagen-induced arthritis, infectious arthritis, Lyme arthritis, proliferative arthritis, psoriatic arthritis, Still's disease, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica spectrum disorder (NMO, also known as Devic's Disease or Devic's Syndrome), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, Type I diabetes, Type II diabetes, latent autoimmune diabetes in adults (or Type 1.5 diabetes) Also contemplated are immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and gianT cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, anti-phospholipid syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopenic purpura (ITP) including chronic or acute ITP, acquired thrombocytopenic purpura, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, gianT cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyly, and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, gianT cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, emphysema, alopecia areata, adipose tissue inflammation/diabetes type II, obesity associated adipose tissue inflammation/insulin resistance, and endometriosis.

GTS-21 is also known as 3-[(3E)-3-[(2,4-dimethoxyphenyl)methylidene]-5,6-dihydro-4H-pyridin-2-yl]pyridine, having the structure:

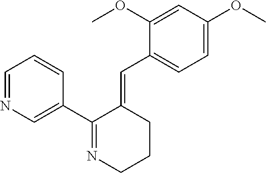 [SMILES: COc1ccc(/C=C2\CCCN=C2c2cccnc2)c(OC)c1]

4-OH-GTS-21 is also known as 3-(4-hydroxy, 2-Methoxybenzylidene)anabaseine (see Meyer E M, et al. “Analysis of 3-(4-hydroxy, 2-Methoxybenzylidene)anabaseine selectivity and activity at human and rat alpha-7 nicotinic receptors”) Journal of Pharmacology and Experimental Therapeutics. 1998 December; 287(3):918-25.

TC-5619 (also known as Bradanicline) has the chemical structure:

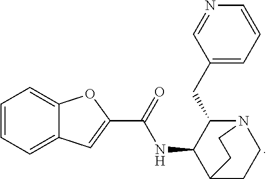 [SMILES: O=C(N[C@@H]1C2CCN(CC2)[C@H]1Cc1cccnc1)c1cc2ccccc2o1]

AZD1446 (also known as 3-(5-Chloro-2-furoyl)-3,7-diazabicyclo[3.3.0]octane, has the chemical as reported in Mazurov, et al. (2012) J. Med. Chem. 55(21), 9181-9194).

TC-6987-23 is disclosed and the method for making it is found in U.S. Pat. Nos. 8,476,296; 8,901,151; 9,173,876 and 8,124,619.

PHA543613 has the chemical structure:

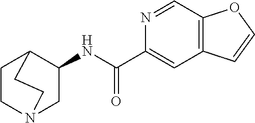 [SMILES: O=C(N[C@H]1CN2CCC1CC2)c1cc2ccoc2cn1]

PHA568487 (CAS 527680-57-5, N-(3R)-1-Azabicyclo[22.2]oct-3-yl-2,3-dihydro-1,4-benzodioxin-6-carboxamide fumarate) is commercially available from R& D systems (Catalog #3134).

PNU282987 N-(3R)-1-Azabicyclo[2.2.2]oct-3-yl-4-chlorobenzamide) is commercially available from Tocris (Catalog No. 2303). It has the chemical structure:

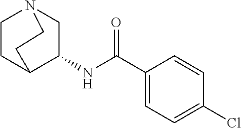 [SMILES: O=C(N[C@H]1CN2CCC1CC2)c1ccc(Cl)cc1]

A “steroid” is an organic compound with four rings arranged in a specific molecular configuration. Non-limiting examples include lipid cholesterol, estradiol, testosterone, and dexamethasone.

A “corticosteroid” is a member of a class of compounds that are produced in the adrenal cortex of vertebrates, as well as the synthetic analogues of these hormones. Non-limiting examples include cortisol, corticosterone, cortisone, and aldosterone.

An antibiotic intends compounds that treat or prevent bacterial infections. Non-limiting examples include penicillins such as penicillin and amoxicillin, cephalosporins such as cephalexin, macrolides such as erythromycin, clarithromycin, and azithromycin, fluoroquinolones such as ciprofloxacin, levofloxacin, and ofloxacin, sulfonamides such as co-trimoxazole and trimethoprim, tetracyclines such as tetracycline and doxycycline, and aminoglycosides such as gentamicin and tobramycin.

An alpha blocker is a pharmacological agent that acts as neutral antagonists of alpha-adrenergic receptors. Non-limiting examples include phenoxybenzamine, phentolamine, tolazoline, trazodone, alfuzosin, doxazosin, prazosin, tamsulosin, terazosin silodosin, atipamezole, idazoxan, mirtazapine and yohimbine.

A beta is a competitive antagonist that block the beta-adrenergic receptors. Non-limiting examples include acebutolol, atenolol, betaxolol, bisoprolol fumarate, carvedilol, esmolol, labetalol, metaprolol, nadlol, nebivolol, penbutolol, pindolol, sotlol, and timolol.

An antihistamine is a drug that inhibits the activity of histimine receptors. Non-limiting examples include azelastine, azelastine, carbinoxamine, cyproheptadine, desloratadine, emedastine, hydroxyzine, and levocabastine.

A blocking antibody against Th2 cytokine intends an antibody or fragment thereof that inhibits Th2 cytokine production.

Immunolgobulin E or “IgE” intends an antibody isotype.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks.

In one aspect, the term “equivalent” means the ability of the chemical, protein, antibody or protein to selectively interact with its target protein, DNA, RNA or fragment thereof as measured by the activation of the target protein, incorporation of the chemical into the DNA or RNA or other suitable methods. Equivalents include, but are not limited to, those agents with the same or similar biological activity and include, without limitation a pharmaceutically acceptable salt or mixtures thereof that interact with and/or inactivate the same target protein, DNA, or RNA as the reference chemical.

### MODES FOR CARRYING OUT THE ASPECTS OF THE DISCLOSURE

This disclosure provides one or more of:
a. reducing ILC2 effector function;
b. repressing IC2-dependent AHR;
c. decreasing expression of ILC2 transcription factor GATA-3;
d. decreasing expression of ILC2 inflammatory modulator NF-κB;
e. reducing phosphorylation of kinase IKKα/β; or
f. reducing ILC2-mediated cytokine production;
   by methods comprising, or alternatively consisting essentially of, or yet further consisting of, contacting an ILC2-cell expressing α7-nicotine acetylcholne receptor (α7nAChr) or a tissue containing such cell, with an effective amount of an α7nAChr agonist, that is optionally contacted in one or more doses. In one aspect, the an α7nAChr agonist comprises an active form of GTS-21, or an equivalent or a pharmaceutically acceptable salt thereof. A non-limiting example of the active form of GTS-21 comprises 4-OH-GTS-21, or a pharmaceutically acceptable salt thereof. In a further aspect, equivalent interacts with the α7-specific residues located at loop C (Arg182 and Glu185) and loop F (Glu158 and Asp160). The method can further comprise contacting the cell with an effective amount of an additional agent selected from the group of: a anti-inflammatory agent, a steroid, a corticosteroid, an antibiotic, an alpha blocker, a beta blocke, an antihistamine, a blocking antibody against Th2 cytokines, an IgE, a LABA, a SABA, a LAMA, and an MABA, to the subject, that is optionally administered concurrently or sequentially in one or more doses. One of skill in the art can determine when the object of the method has been met by assaying for the recited function in vitro using methods known in the art and as disclosed herein.

The method can be practiced in vitro or in vivo, as exemplified herein and the ILC2 is an animal cell or a mammalian cell, non-limiting examples of such include a human cell, a canine cell, a murine cell, a feline cell, a bovine cell or an equine cell.

Also provided herein is a method for one or more of
a. reducing ILC2 effector function;
b. repressing IC2-dependent AHR;
c. decreasing expression of ILC2 transcription factor GATA-3;
d. decreasing expression of ILC2 inflammatory modulator NF-κB;
e. reducing phosphorylation of kinase IKKα/β;
f. reducing ILC2-mediated cytokine production;
g. treating and/or ameliorating AHR;
h. treating and/or ameliorating allergic inflammation; oe
i. treating and/or ameliorating an inflammatory condition,
   in a subject in need thereof, comprising, or alternatively consisting essentially of, or yet further consisting of administering to the subject an effective amount of effective amount of an α7nAChr agonist. In one aspect, the an α7nAChr agonist comprises an active form of GTS-21, or an equivalent or a pharmaceutically acceptable salt thereof. In another aspect, the active form of GTS-21 comprises 4-OH-GTS-21, or a pharmaceutically acceptable salt thereof. A non-limiting example of an equivalent is a compound or other active agent (e.g. antibody or peptide) that interacts with the α7-specific residues located at loop C (Arg182 and Glu185) and loop F (Glu158 and Asp160). The method can further comprise administering to the subject an effective amount of an additional agent selected from the group of: an anti-inflammatory agent, a steroid, a corticosteroid, an antibiotic, an alpha blocker, a beta blocke, an antihistamine, a blocking antibody against Th2 cytokines, an IgE, a LABA, a SABA, a LAMA, and an MABA, to the subject, that is optionally administered concurrently or sequentially in one or more doses. One of skill in the art can determine when the object of the method has been met by assaying for the recited function in vivo using methods known in the art and as disclosed herein.

In one aspect, the subject is an animal or a human. When the subject is a non-human animal, the method is useful to screen for additional or complementary agents for administration. In a further aspect, the mammal is of the group of a human, a canine, a murine, a feline, a bovine, or an equine.

Further provided are methods for treating or ameliorating the symptoms of a disease or condition related to ILC2 effector function in a subject, or for treating or ameliorating an inflammatory condition or disease in a subject need thereof, the method comprising administering an effective amount of an α7nAChr agonist to the subject. In one aspect the α7nAChr agonist is GTS-21, or an equivalent or a pharmaceutically acceptable salt thereof. Non-limiting examples of a disease or condition related to ILC2 effector function include inflammatory disorders such as acute and chronic inflammatory disorders, rheumatoid arthritis, COPD, IBS, Crohn's disease, ulcerative colitis, a food allergy, allergic asthma, non-allergic asthma, lung inflammation, mucosal inflammation, nasal polyps, eczema, and atopic dermatitis.

In one aspect, the disese or condition is COPD or asthma. The α7nAChr agonist is GTS-21, or an equivalent or a pharmaceutically acceptable salt thereof is combined with one or more of the following agents in a single or separate administration as determined by the treating physician: LABA (Long-acting beta-agonist bronchodilators, such as bambuterol, clenbuterol, formoterol, salmeterol, carmoterol, milveterol, vilanterol, indacaterol, saligenin- or indole-containing and adamantyl-derived β2 agonists, and pharmaceutically acceptable salts, esters, or isomers thereof; SABA (short-acting beta-agonist bronchodilators) such as salbutamol (albuterol), levalbuterol, pirbuteroi, terbutaline, epinephrine, or a pharmaceutically acceptable salts thereof; LAMA (long-acting muscarinic antagonist), such as tiotropium bromide, glycopyrronium bromide; aclidinium bromide, umeclidinium bromide, and pharmaceutically acceptable salts, esters, or isomers thereof; muscarinic antagonists, and pharmaceutically acceptable salts, esters, or isomers thereof, examples of such include without limitation atropine, hyoscyamine, hyoscine butylbromide, hydrobromide, ipratropium, tropicamide, cyclopentolate, and pirenzepine (additonal examples can be found at the web address en.wikipedia.org/wiki/Muscarinic_antagonist); inhaled corticosteroids such as beclomethasone diproprionate, fluticasone furoate or fluticasone proprionate, ciclesonide, mometasone furoate, budesonide, flunisolide, or triamcinolone or a mixture thereof, and pharmaceutically acceptable salts, esters, or isomers thereof; MABA (Bifunctional Muscarinic Antagonist-Beta2 Agonist), such as GSK961081, CHF6366, AZD8999(LAS190792), AZD8871, and pharmaceutically acceptable salts, esters, or isomers thereof.

In one aspect, the α7nAChr agonist, e.g., GTS-21 or a pharmaceutically acceptable salt thereof is coadmininistered with an effective amount of an anti-inflammatory identified herein, such as a steroid or corticosteroid, or an antibiotic, an alpha or beta blocker, an antihistamine, a blocking antibody against Th2 cytokine and IgE. These can be administered to reduce inflammation, mucosal inflammation, nasal polyps, eczema, and atopic dermatitis. Formulations containing the active ingredients are further provided herein.

In a further aspect, the α7nAChr agonist such as GTS-21, or an equivalent or a concurrently or sequentially. In one aspect, an effective amount of the α7nAChr agonist such as GTS-21, or an equivalent or a pharmaceutically acceptable salt thereof is combined with an effective amount of the anti-inflammatory such as a steroid or corticosteroid, or antibiotics, alpha and beta blockers, antihistamine, blocking antibodies against Th2 cytokines and IgE, in a single formulation for simultaneous administration.

For the purpose of the method, the subject is an animal or a mammal, e.g., a human, a canine, a murine, a feline, a bovine or an equine.

In one aspect, the compostions as described herein treat and/or ameliorating disease or conditions are administered in a formulation appropriate for the disease and subject. For example to treat and/or ameliorate AHR, the agents are administered by inhalation thereby targeting respiratory inflammation; or as a gum, syrup or cream for upper respiratory inflammation including nasal polyps, soar throats and cough (upper respiratory inflammation). Alternatively they can be administered as a topic formulation such as a hand cream for eczema and dermatitis; or as an oral administration to target upper and lower GI tract inflammation, including IBD and food allergy or systemic or local administration to target systemic or local inflammation related to ILC2s and other immune cells. For the treatment and/or amelioration of rheumatoid arthritis, systemic or local administration is used to target systemic or local inflammation related to ILC2s and other immune cells.

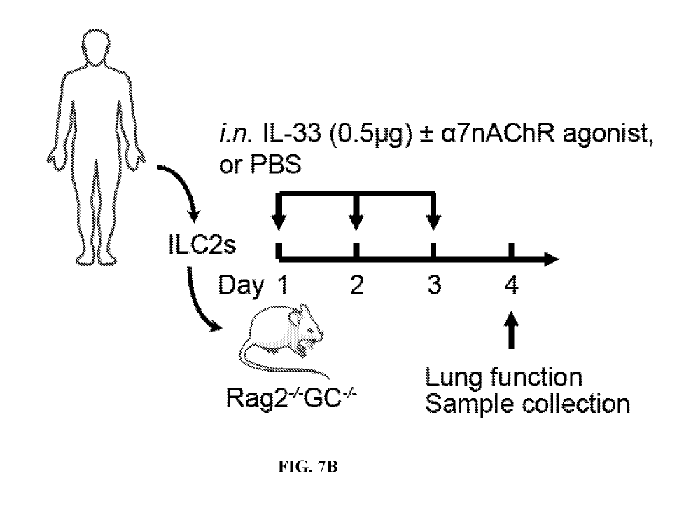

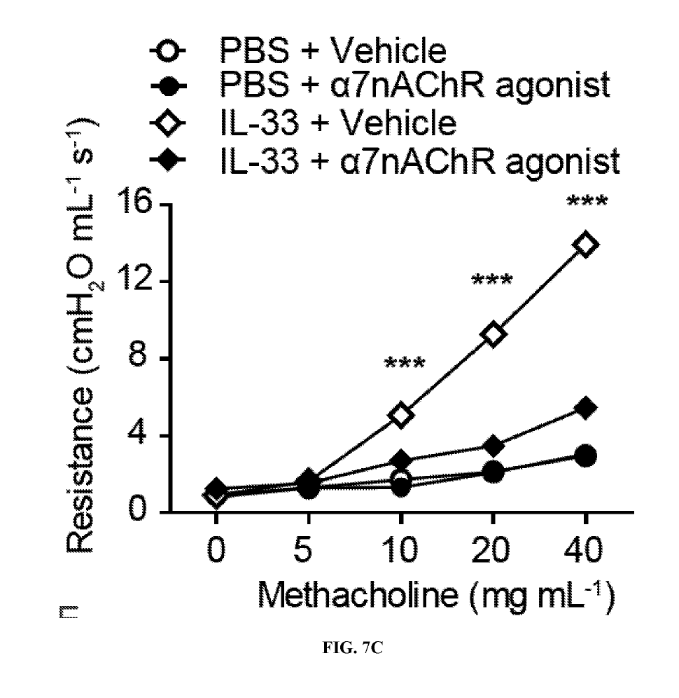

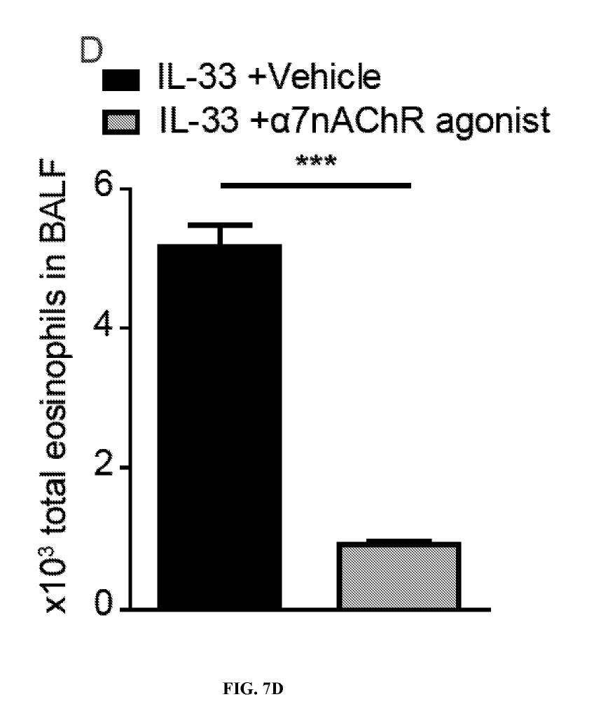

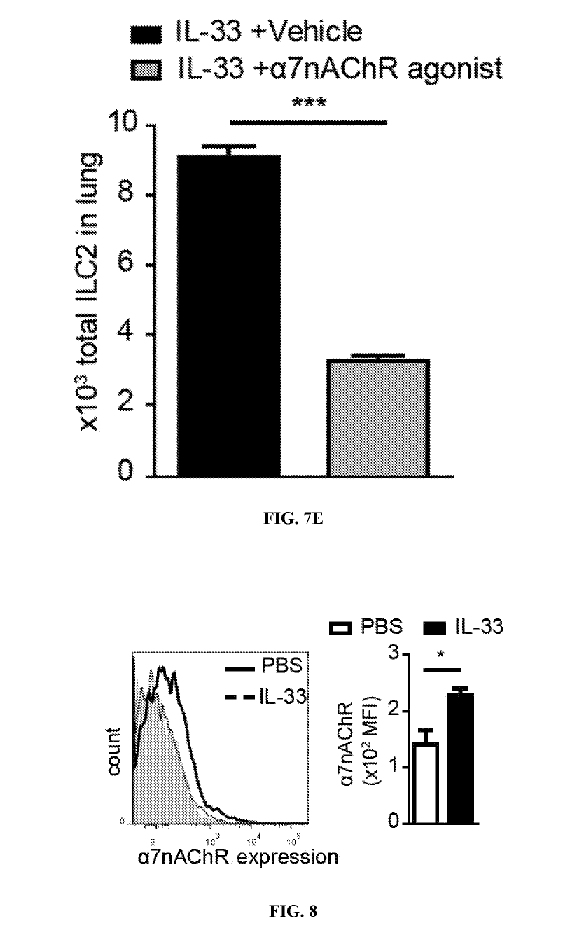

Applicant's results as shown herein provide evidence that the α7nAChr agonist such as GTS-21, or an equivalent major, provides the therapeutic benefit as claimed. A major breakthrough in the generation of humanized mice was the development of immunodeficient mice bearing a targeted IL-2Rc mutation. These mice permit functional in vivo studies of human cells and tissues. Models have been developed in which immunodeficient alymphoid mice are ‘humanized’, by adoptive transfer of sorted human cells such as ILC2s (see for example FIG. 7B of Galle-Treger et al. (2016). Nicotinic acetylcholine receptoragonist attenuates ILC2-Dependent airway hyperreactivity. Nat. Commun. 7, 13202, doi: 10.1038/ncomms13202). These animals can be challenged with allergens or other stimuli, after which disease phenotypes, such as airway hyperreactivity (AHR), inflammation and eosinophilia, can be measured. This system allows the study the cellular or molecular pathways that target ILC2s and ILC2 related diseases, including inflammatory disorders such as upper respiratory inflammation, lower respiratory inflammation, rhinitis, sinusitis, allergic and non allergic asthma, eczema, atopic dermatitis, rheumatological diseases including rheumatoid arthritis and finally diseases related to main ILC2 enriched region including mucosal tissues and intestinal tract (inflammatory bowel diseases (IBD), including Crohn's disease (CD) and ulcerative colitis (UC) and food allergy.

Further provided herein are compositions comprising an α7nAChr agonist as described herein and anti-inflammatory such as a steroid or corticosteroid, or antibiotics, alpha and beta blockers, antihistamine, blocking antibodies against Th2 cytokines and IgE. In a further aspect, an effective amount (e.g., a therapeutically effective amount) is provided in the formulation. The formulation is prepared for appropriate administration, e.g., locally or systemically, topically, by inhalation, ingestion (orally), or by infusion.

Also provided herein is a method for screening for an α7nAChr agonist, comprising, or alternatively consisting essentially of, or yet further consisting of, contacting an ILC2 cell expressing α7nAChr or tissue sample comprising ILC2 cells expressing α7nAChr, with a test agent and evaluating for the cell or tissue for one or more of:
a. reducing ILC2 effector function;
b. decreasing expression of ILC2 transcription factor GATA-3;
c. decreasing expression of TLC2 inflammatory modulator NF-κB;
d. reducing phosphorylation of kinase IKKα/β;
e. reducing ILC2-mediated cytokine production;
f. treating and/or ameliorating AHR;
g. treating and/or ameliorating allergic inflammation; or
h. treating and/or ameliorating an inflammatory condition or response related to ILC-2 effector function.
   wherein a measured response of a. to h. identifies the test agent as a possible α7nAChr agonist. One of skill in the art can determine when the object of the method has been met by assaying for the recited function in vitro or in vivo using methods known in the art and as disclosed herein.

In one aspect, the method further comprises comparing the activity of the test agent with the α7nAChr agonist activity of 4-OH-GTS-21. The method can further comprise contacting the cell or tissue or by administering to the subject an effective amount of an additional agent selected from the group of: an anti-inflammatory agent, a steroid, a corticosteroid, an antibiotic, an alpha blocker, a beta blocke, an antihistamine, a blocking antibody against Th2 cytokines, an IgE, a LABA, a SABA, a LAMA, and an MABA, that is optionally contacted or administered concurrently or sequentially in one or more doses or administrations.

The contacting can be in vitro or in vivo, and/or the ILC2 is an animal cell or a mammalian cell, non-limiting examples of such include a human cell, a canine cell, a murine cell, a feline cell, a bovine cell or an equine cell.

Further provided herein is a method for screening for an agent that treats or ameliorates ILC2-mediated AHR and/or allergic inflammation and/or inflammation, comprising, or alternatively consisting essentially of, or yet further consisting of: administering to a subject suffering ILC2-mediated AHR and/or allergic inflammation an amount of ILC2-mediated AHR and/or allergic inflammation, wherein a measured response identifies the test agent as a possible α7nAChr agonist. In one aspect, the method further comprises comparing the activity of the test agent with the α7nAChr agonist activity of 4-OH-GTS-21. The method can further comprise contacting the cell or tissue or by administering to the subject an effective amount of an additional agent selected from the group of: a anti-inflammatory agent, a steroid, a corticosteroid, an antibiotic, an alpha blocker, a beta blocke, an antihistamine, a blocking antibody against Th2 cytokines, an IgE, a LABA, a SABA, a LAMA, and an MABA, that is optionally contacted or administered concurrently or sequentially in one or more doses or administrations. One of skill in the art can determine when the object of the method has been met by assaying for the recited function in vivo using methods known in the art and as disclosed herein. The subject is of the group of: a human, a canine, a murine, a feline, a bovine or an equine.

The agonist compounds of the technology can be administered by parenteral (e.g., intramuscular, intraperitoneal, intravenous, ICV, intracisternal injection or infusion, subcutaneous injection, or implant), oral, by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration.

### Compositions and Formulations

In one embodiment, this technology relates to a composition comprising a compound as described herein and a carrier.

In another embodiment, this technology relates to a pharmaceutical composition comprising a compound as described herein and a pharmaceutically acceptable carrier.

In another embodiment, this technology relates to a pharmaceutical composition comprising a therapeutically effective amount of a compound as described herein and a pharmaceutically acceptable carrier.

The pharmaceutical compositions for the administration of the compounds can be conveniently presented in dosage unit form and can be prepared by any of the methods well known in the art of pharmacy. The pharmaceutical compositions can be, for example, prepared by uniformly and intimately bringing the compounds provided herein into association with a liquid carrier, a finely divided solid carrier or both, and then, if necessary, shaping the product into the desired formulation. In the pharmaceutical composition the compound provided herein is included in an amount sufficient to produce the desired therapeutic effect. For example, pharmaceutical compositions of the technology may take a form suitable for virtually any mode of administration, including, for example, topical, ocular, oral, buccal, systemic, nasal, injection, infusion, transdermal, rectal, and vaginal, or a form suitable for administration by inhalation or insufflation.

For topical administration, the compounds can be formulated as solutions, gels, ointments, creams, suspensions, etc., as is well-known in the art.

Systemic formulations include those designed for administration by injection (e.g., subcutaneous, intravenous, infusion, intramuscular, intrathecal, or intraperitoneal injection) as well as those designed for transdermal, transmucosal, oral, or pulmonary administration.

Useful injectable preparations include sterile suspensions, solutions, or emulsions of the compounds provided herein in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, and dextrose solution, before use. To this end, the compounds provided herein can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the compounds provided herein in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g. starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques well known to the skilled artisan. The pharmaceutical compositions of the technology may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

### Kits

The compounds and compositions, as described herein, can be provided in kits. The kits can further contain additional dUTPase inhibitors and optionally, instructions for use. In a further aspect, the kit contains reagents and instructions to perform the screen to identify patients more likely to respond to the therapy as described above.

### Experimental

α7nAChR Agonists and α7nAChR Expression by ILC2s

Allergic asthma is a complex and chronic inflammatory disorder that is associated with airway hyperreactivity (AHR) and driven by Th2 cytokine secretion. Type 2 innate lymphoid cells (ILC2s) produce large amounts of Th2 cytokines and contribute to the development of AHR. Here, Applicants show that ILC2s express the α7-nicotinic acetylcholine receptor (α7nAChR), which is thought to have an anti-inflammatory role in several inflammatory diseases. Applicants show that engagement of a specific agonist with α7nAChR on ILC2s reduces ILC2 effector function and represses ILC2-dependent AHR, while decreasing expression of ILC2 key transcription factor GATA-3 and critical inflammatory modulator NF-κB, and reducing phosphorylation of upstream kinase IKKα/β. Additionally, the specific α7nAChR agonist reduces cytokine production and AHR in a humanized ILC2 mouse model. Collectively, our data suggest that α7nAChR expressed by ILC2s is a potential therapeutic target for the treatment of ILC2-mediated asthma.

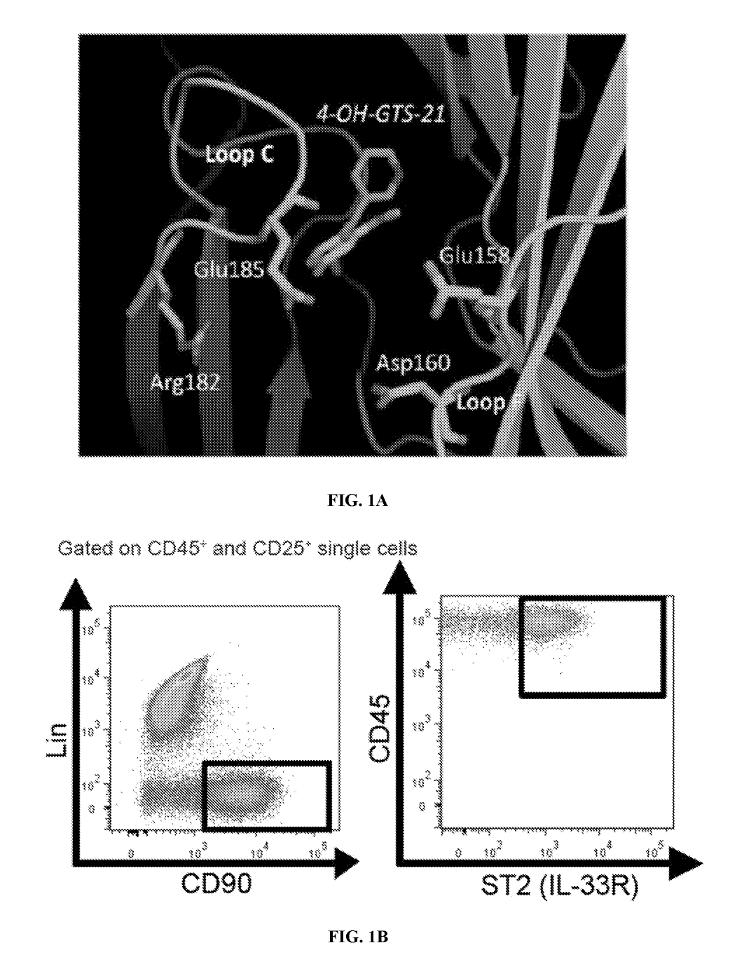

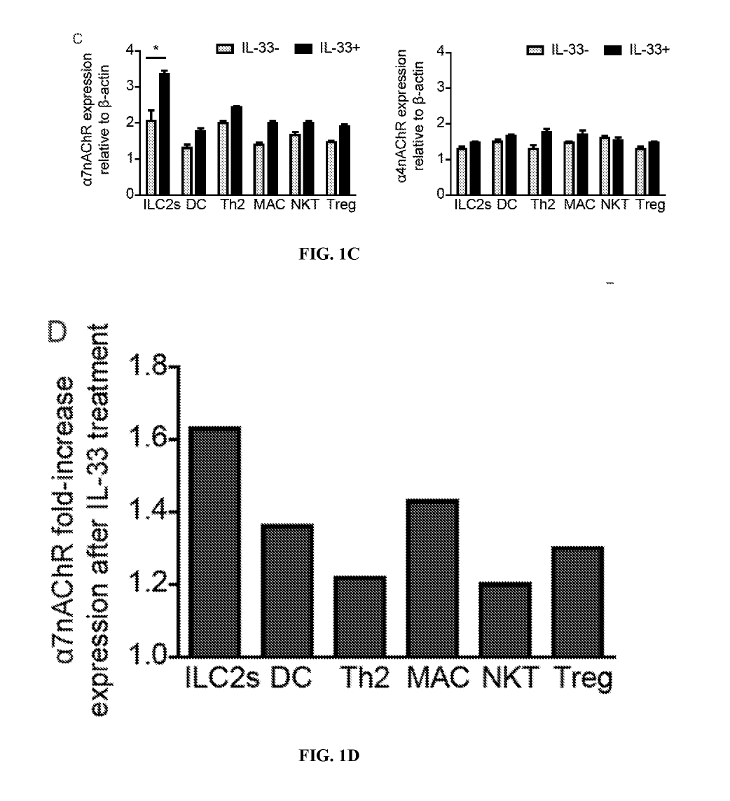

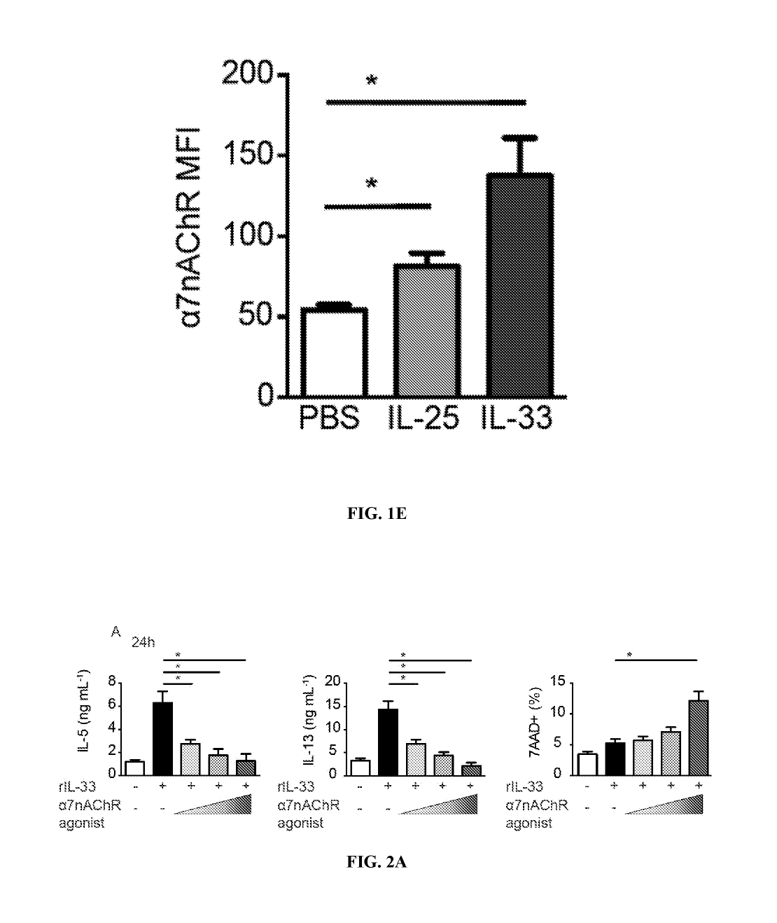

Because of the potential therapeutic application in neurological disorders and inflammation, α7-selective ligands have been intensively studied. Li et al.²¹ solved the crystal structure of a receptor chimera constructed from the extracellular domain of α7nAChR and acetylcholine binding protein (AChBP), which represents the closest structural homolog of the native α7nAChR. Applicants have also determined structures of α7nAChR bound by agonist and antagonist^{21,26}, and performed structure-based screens of α7nAChR-specific compounds²². These studies, together with those reported by others, have demonstrated that the crystal structure of the α7nAChR/AChBP chimera is a much better template for structure-based drug screening than AChBP²³, which has been widely used in previous drug screening and design. These studies have identified a variety of potential ligands and allosteric modulators of α7nAChR, including many previously known α7-specific compounds. Of these, GTS-21 (DMXB-A) has been previously characterized as a neuronal nAChR ligand. GTS-21 binds to both the α4β2 and α7 subtypes^{24,25,27,28}, but activates only the α7 subtype to a significant extent. In this structure-based drug screen, Applicants selected 4-OH-GTS-21, the active form of GTS-21 in vivo, from the docking analyses. As shown in FIG. 1A, the overall structure is similar to that of 4-OH-GTS-21 bound to AChBP²⁹, including the conformation and orientation of 4-OH-GTS-21 bound to the ligand pocket. On the other hand, Applicants' docking analyses reveal that a number of α7-specific residues located at loop C (Arg182 and Glu185) and loop F (Glu158 and Asp160), which are absent in AChBP, interact, or are poised to interact, with functional groups on 4-OH-GTS-21. These observations provide structural bases to guide further synthetic modification of GTS-21 to gain higher selectivity toward the α7 subtype and to modify the functional effects of similar ligands, thus expanding the therapeutic repertoire of agents based on the anabaseine scaffold.

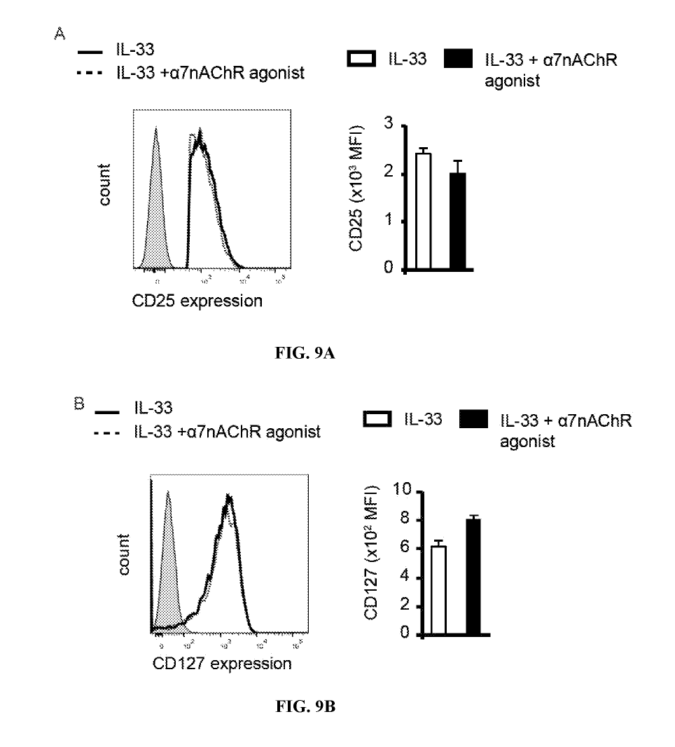

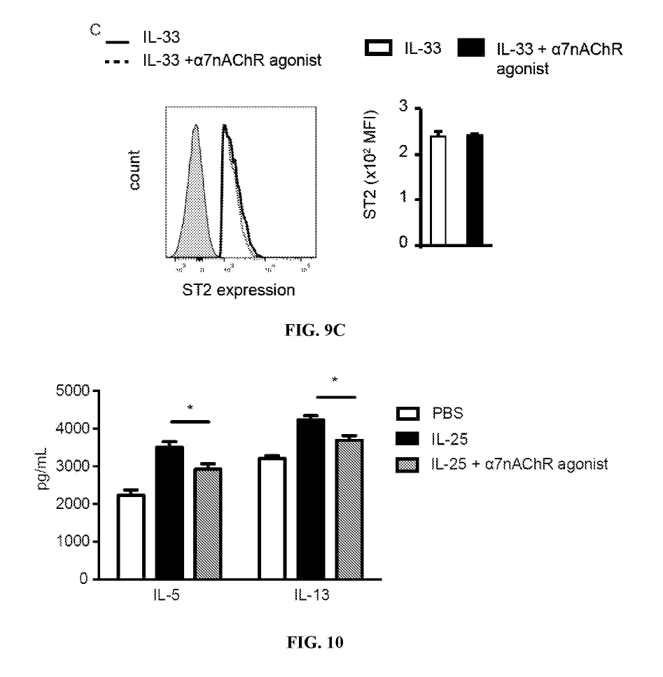

The α7nAChR is present on macrophages¹⁵, T cells^{30,31} and B cells³², and activation of this receptor mediates anti-inflammatory effects. However, there are no studies investigating α7nAChR expression on ILC2s, despite their essential role in the development of asthma. Applicants identified ILC2s as a Lin⁻, CD25⁺, CD45⁺, CD90.2⁺, and ST2⁺ population (FIG. 1B). As mentioned previously, ILC2s are activated by IL-33; however, they are not the only cells that express the IL-33 receptor. As GTS-21 binds to both α4 and α7 subtypes of nAChR, Applicants further assessed mRNA levels of α7 and α4nAChR in various types of immune cells. Interestingly, α7nAChR expression was significantly higher in ILC2s as compared to other ST2⁺ peripheral immune cells (FIG. 1C). Importantly, only α7nAChR in ILC2s, but not α4nAChR, was significantly present and up-regulated by in vitro IL-33 treatment (FIGS. 1C-1D). To confirm this finding at the protein level and also to investigate the effect of IL-25 stimulation, Applicants treated mice with intranasal recombinant mouse (rm)-IL-33, (rm)-IL-25, or PBS as a negative control, for three consecutive days. As shown in FIG. 1E, Applicants found, for the first time, that α7nAChR is expressed on ILC2s. Importantly, α7nAChR expression on ILC2s was significantly up-regulated in mice treated with IL-25 or IL-33, when compared to the PBS control group. α7nAChR expression was additionally confirmed by cytometry using fluorochrome-conjugated α-bungarotoxin, a nicotinic cholinergic blocker (FIG. 8). Meanwhile, α7nAChR did not alter expression of CD25, CD127, and ST2, known as IL-2R, IL-7R, and IL-33R, respectively, and which are essential for development of immune cells (FIG. 9).

α7nAChR Agonist Suppresses Cytokine Production in ILC2s

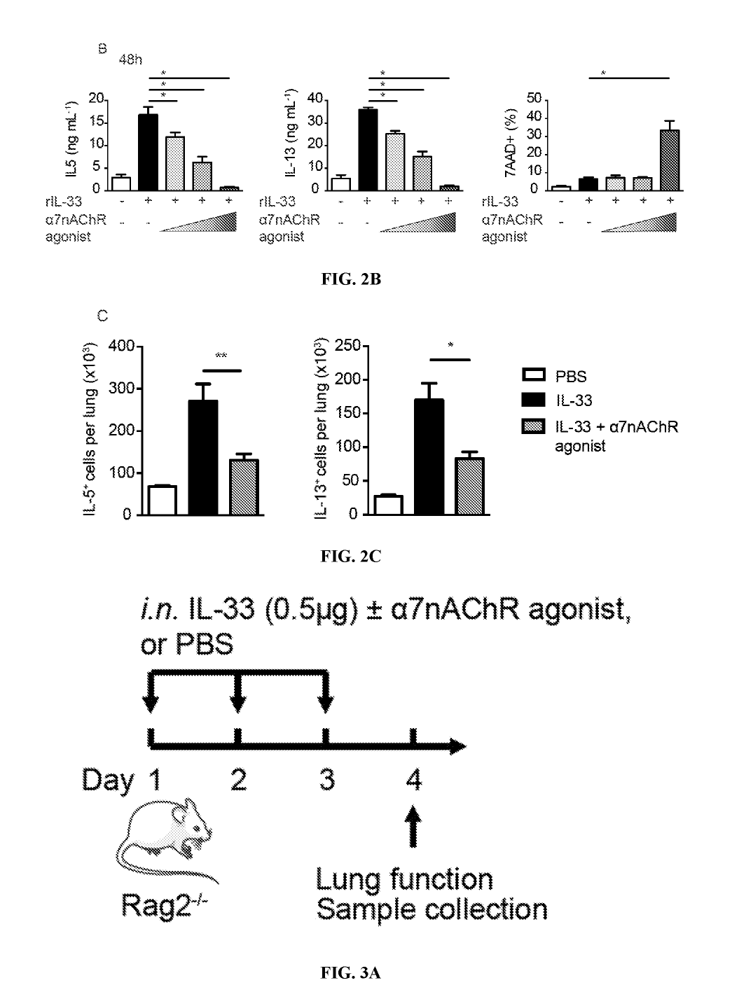

Nicotine is known as a major constituent of cigarette smoke, which causes impairment of lung function and exacerbation of asthma. Interestingly, nicotine administration attenuates production of Th2 cytokines and leukotrienes in preclinical models of asthma¹⁷. Nicotine is an agonist for a variety of pentameric nAChRs made up of different combinations of the sixteen nicotinic receptor subunits, thus it lacks specificity for α7nAChR^{21,33}. Therefore, using GTS-21, an agonist specific for α7nAChR, Applicants sought to determine whether engagement of α7nAChR would alter ILC2s' function. To address this question quantitatively, pulmonary ILC2s were isolated and cultured with increasing doses (2.5, 10 and 50 μg mL⁻¹) of α7nAChR agonist in the presence of rm-IL-33, rm-IL-2 and rm-IL-7. The results show dose-dependent anti-inflammatory effects of α7nAChR agonist on ILC2s (FIG. 2). These effects were independent of cell viability except at the highest tested dose of the agonist (FIGS. 2A-2B, right panels). To verify that the actions of the agonist were not due to a reduction in the number of ILC2s, Applicants administered IL-33 to mice in vivo with or without agonist treatment, and then quantified the number of IL-5⁺ and IL-13⁺ ILC2s within a determined number of ILC2s (FIG. 2C). Similarly to IL-33, IL-25 increased IL-5 and IL-13 secretion in ILC2s in vitro, though this effect was less robust. IL-25-induced cytokine secretion was also susceptible to suppression by the agonist (FIG. 10).

α7nAChR Agonist Treatment Ameliorates ILC2-Induced AHR

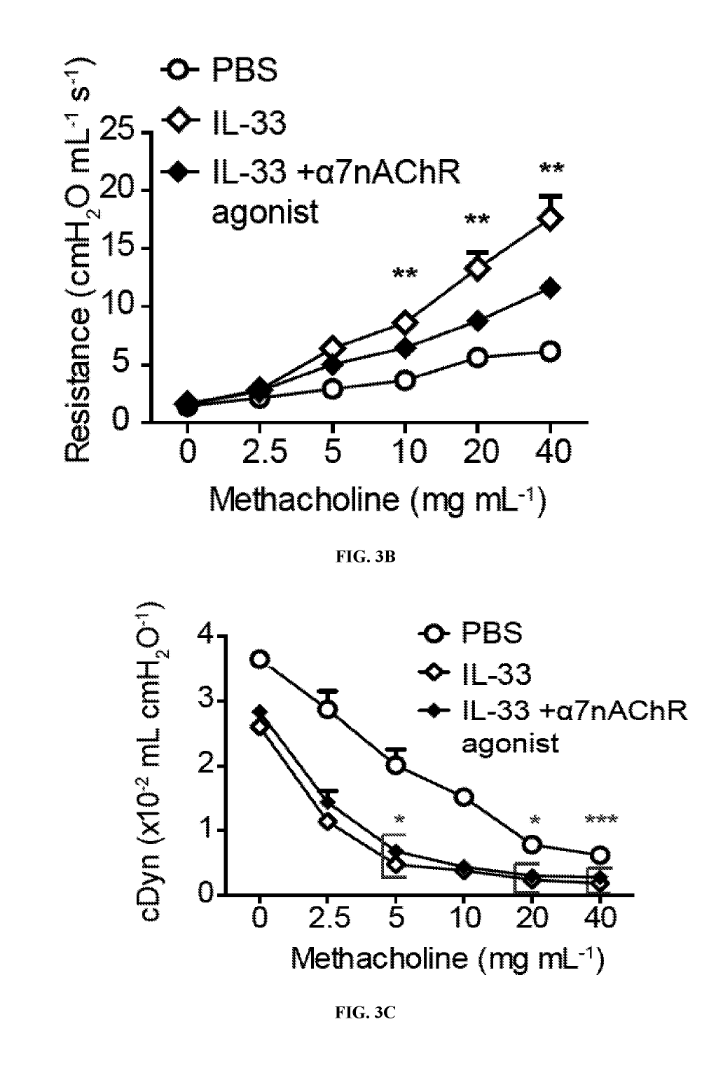

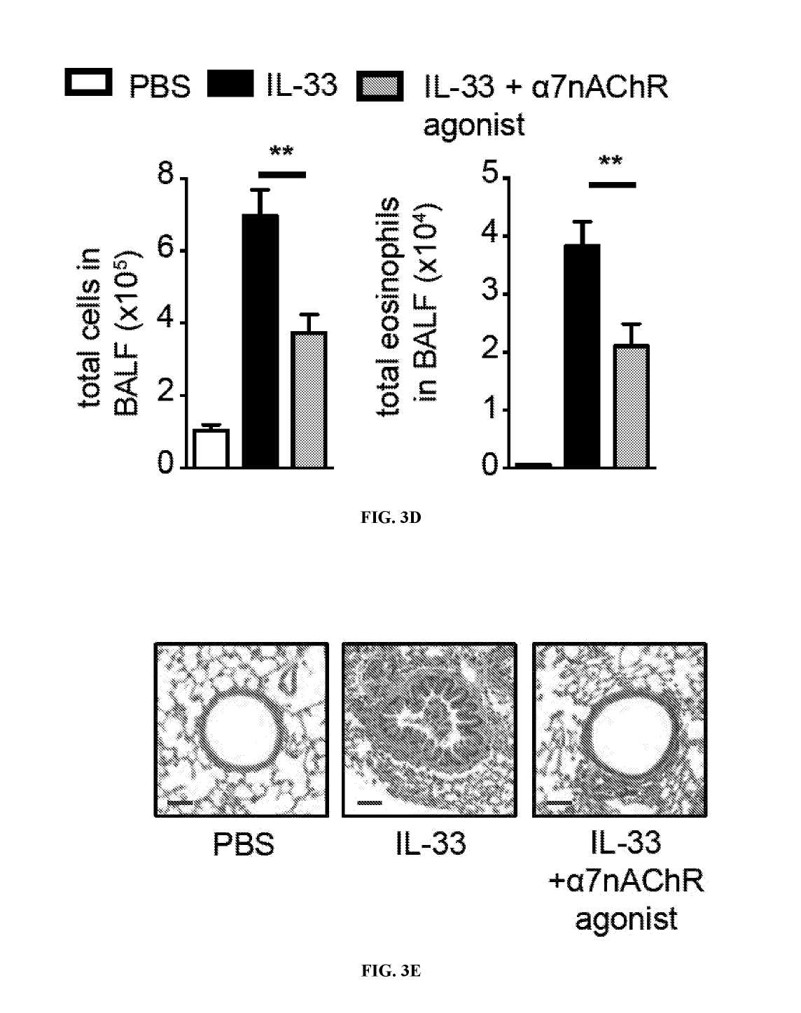

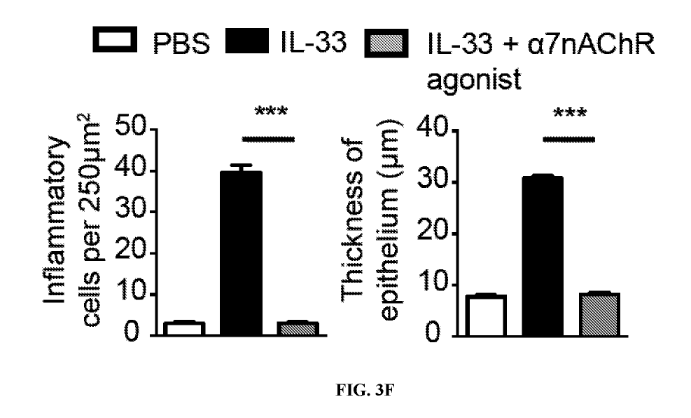

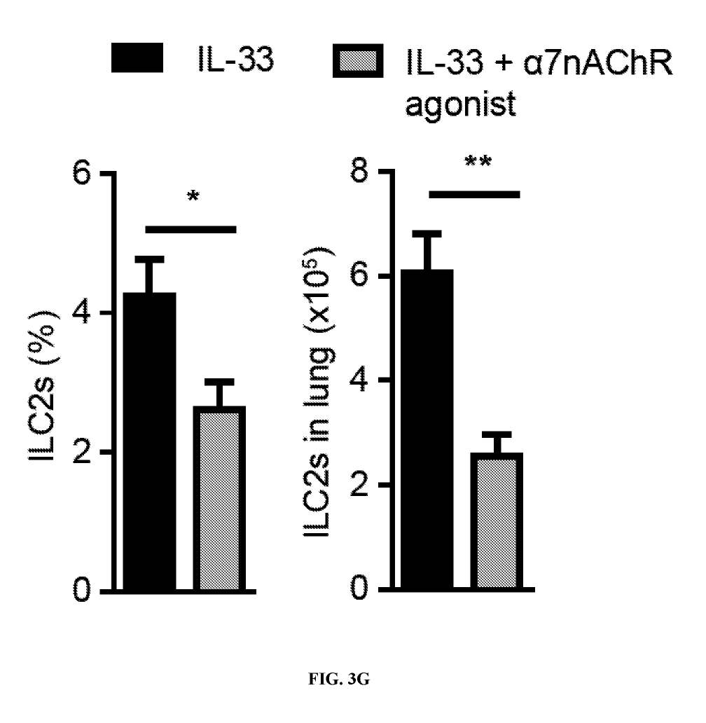

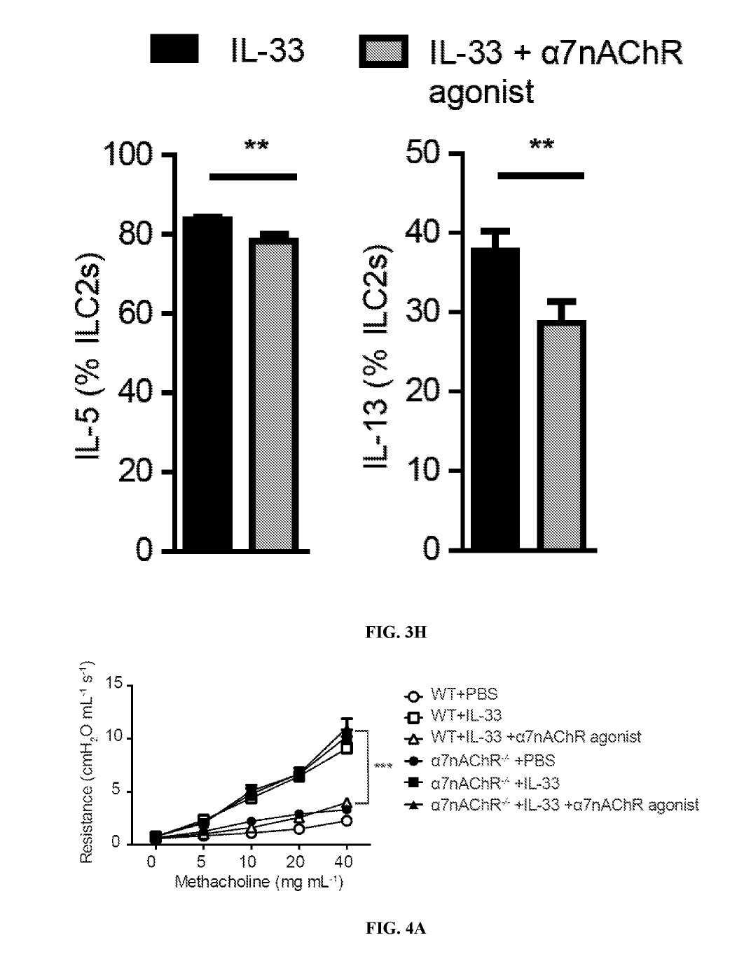

As reported previously^{6-9,11,12,34,35}, the IL-5 and IL-13 cytokines produced by activated ILC2s are essential for eosinophilic inflammation and AHR development. Applicants investigated whether the attenuated ILC2 function by α7nAChR stimulation in vitro could result in inhibition of ILC2-mediated AHR and allergic inflammation. Rag2 deficient mice (devoid of T and B cells) were given intranasal (i.n.) rm-IL-33, with or without α7nAChR agonist for three consecutive days (FIG. 3A). As IL-33 administration specifically induces ILC2s, thereby causing AHR, with this model Applicants can readily explore the effect of an α7nAChR agonist in ILC2-mediated AHR. One day after the last challenge, lung function was evaluated by direct measurements of lung resistance (R_{L}) and dynamic compliance (C_{dyn}), as described in the materials and methods section. Applicants found that stimulating α7nAChR significantly reduced levels of R_{L} and of C_{dyn} in response to IL-33, as compared to PBS (FIGS. 3B-3C). Bronchoalveolar lavage fluid (BALF) analyses also showed decreased eosinophilic infiltration, as well as total cell counts (TCC), in Rag2^{-/-} mice treated with the α7nAChR agonist (FIG. 3D). Histological analyses revealed that α7nAChR stimulation prevented airway wall thickness and infiltrated cells (FIGS. 3E-3F). As shown in FIG. 3G, treatment with GTS-21 also significantly suppressed the frequency and absolute number of lung ILC2s. Furthermore, the intracellular cytokine assay also revealed significantly decreased levels of IL-5 and IL-13 producing lung ILC2s in α7nAChR agonist treated mice compared to untreated mice (FIG. 3H). These findings concur with the reduction of AHR and eosinophil counts in BALF (FIGS. 3B-3D). Thus, α7nAChR agonist represses IL-5 and IL-13 production, eosinophil recruitment, and ILC2-dependent AHR.

α7nAChR on ILC2s is Critical for GTS-21 Action In Vivo

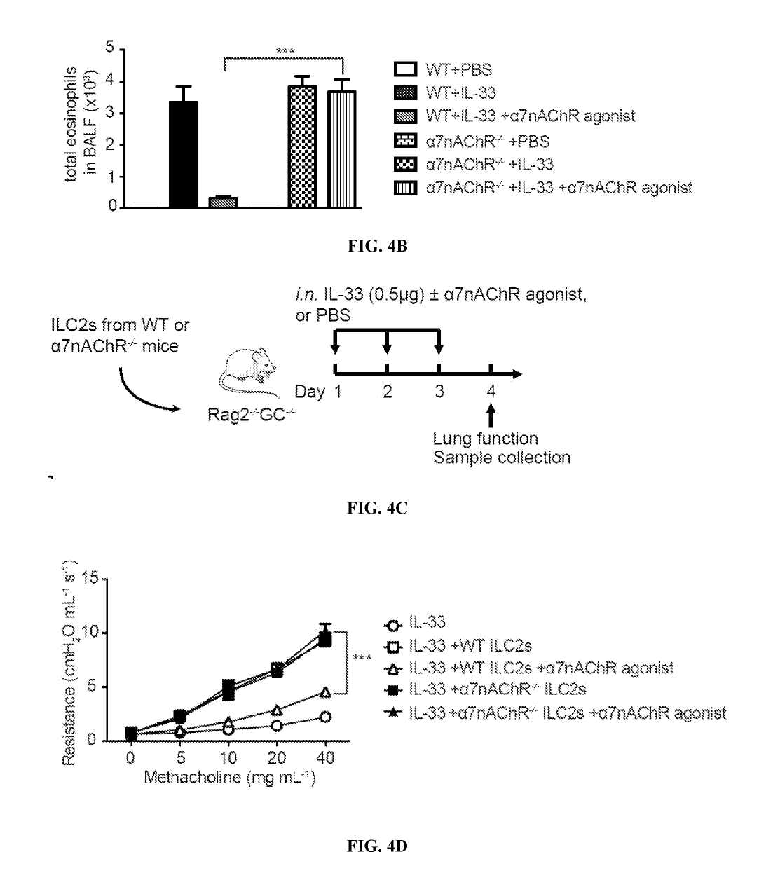

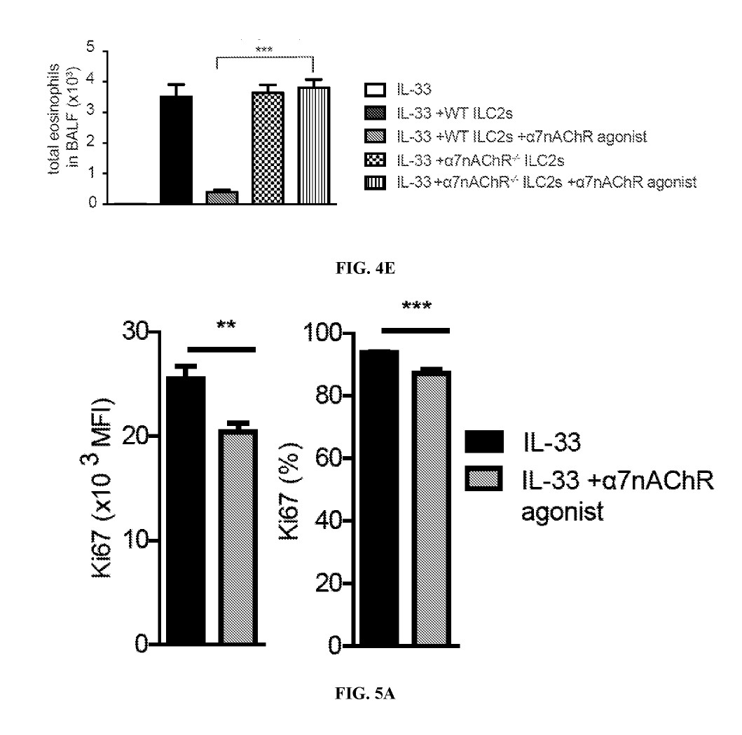

To assess whether the previously observed effects of GTS-21 are due to its actions on the α7nAChR, Applicants challenged WT and α7nAChR-deficient mice with or without rm-IL-33, and with or without α7nAChR agonist, for three consecutive days (FIGS. 4A-4B). As expected, in the WT mice, the agonist repressed IL-33-induced AHR and eosinophilic infiltration. However, in the absence of α7nAChR, the agonist affected neither AHR nor eosinophilia. Taken together, these results indicate that engagement of α7nAChR ameliorates ILC2-mediated AHR and allergic inflammation.

Given that a variety of cells participate in allergic inflammation, Applicants wanted to investigate the effect of the agonist specifically on ILC2s. Rag2^{-/-} GC^{-/-} mice lack not only B and T cells, but NK cells and ILC2s as well. Using methods previously described by our laboratory³⁶, Applicants adoptively transferred WT or α7nAChR-deficient ILC2s into these Rag2^{-/-} GC^{-/-} mice, and then treated them with IL-33, with or without α7nAChR agonist. As expected, Applicants observed a decrease of AHR and of eosinophil recruitment in mice injected with WT ILC2s in response to α7nAChR treatment (FIGS. 4C-4E). However, in the absence of α7nAChR expression on ILC2s, the agonist affected neither AHR nor eosinophilia. Taken together, these results indicate that engagement of α7nAChR ameliorates ILC2-mediated AHR and allergic inflammation. These results, which are consistent with those seen in WT and α7nAChR-deficient mice, demonstrate that the effects Applicants observed are due to ILC2s as opposed to other cells, such as structural cells, which express α7nAChR.

α7nAChR Agonist Attenuates GATA3 and NF-κB Expression

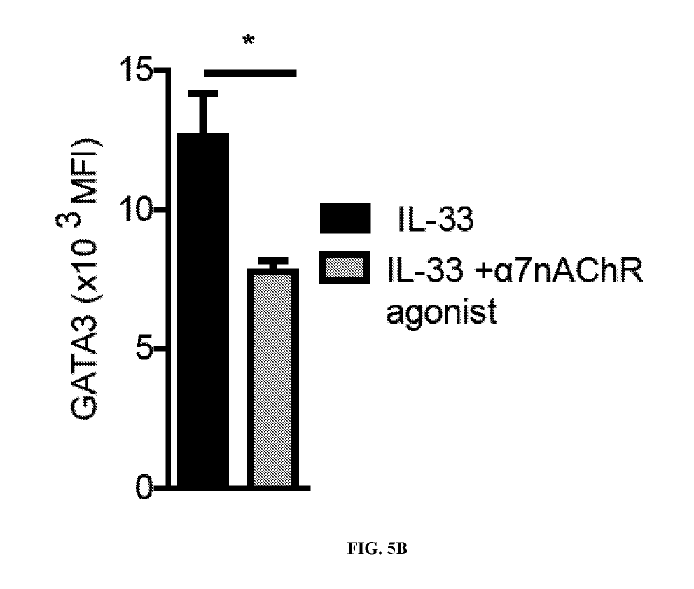

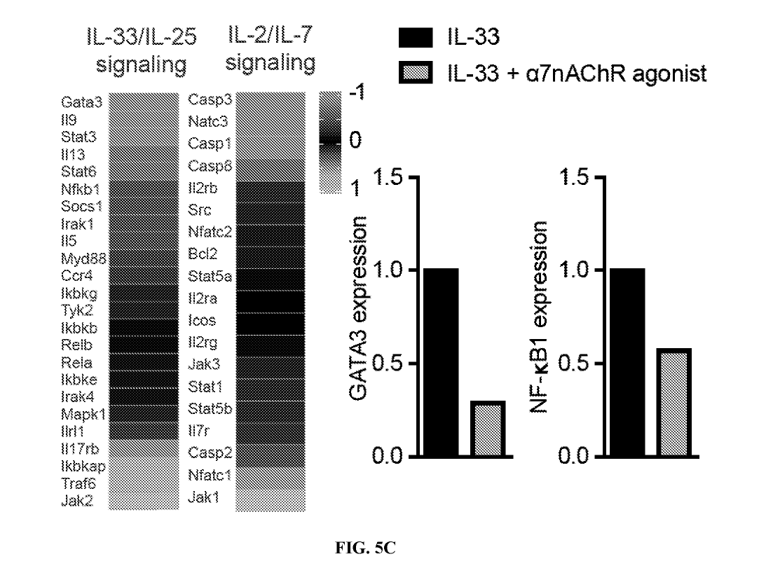

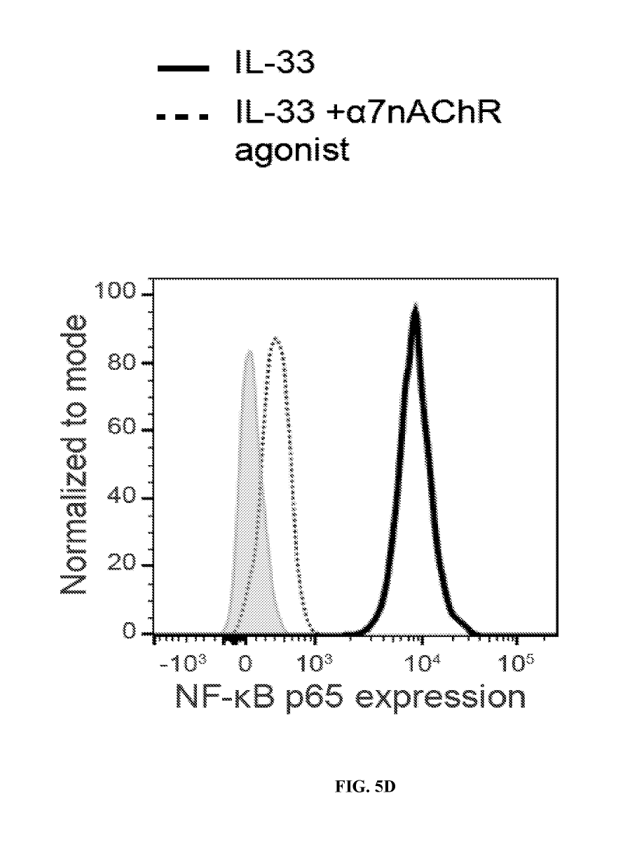

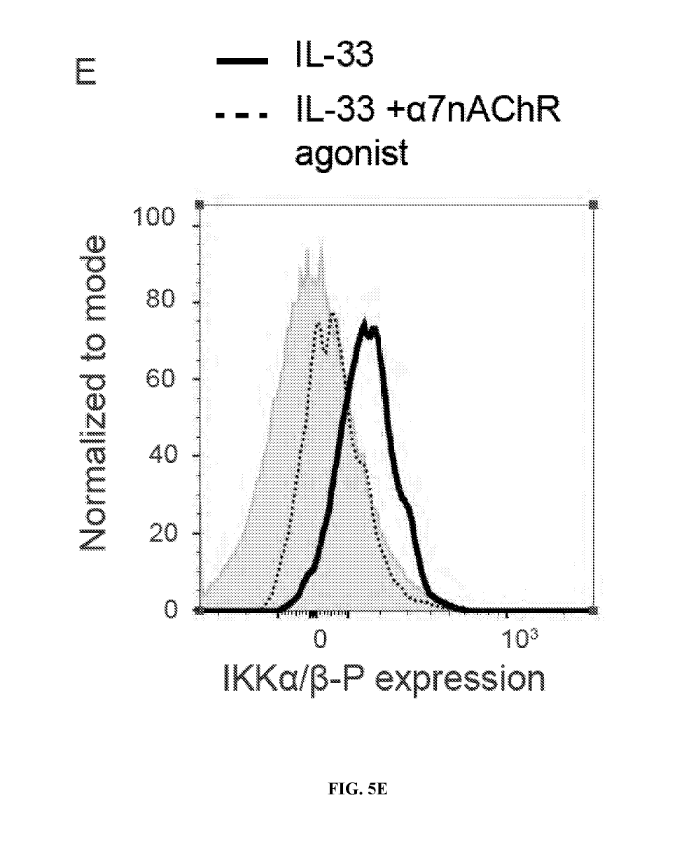

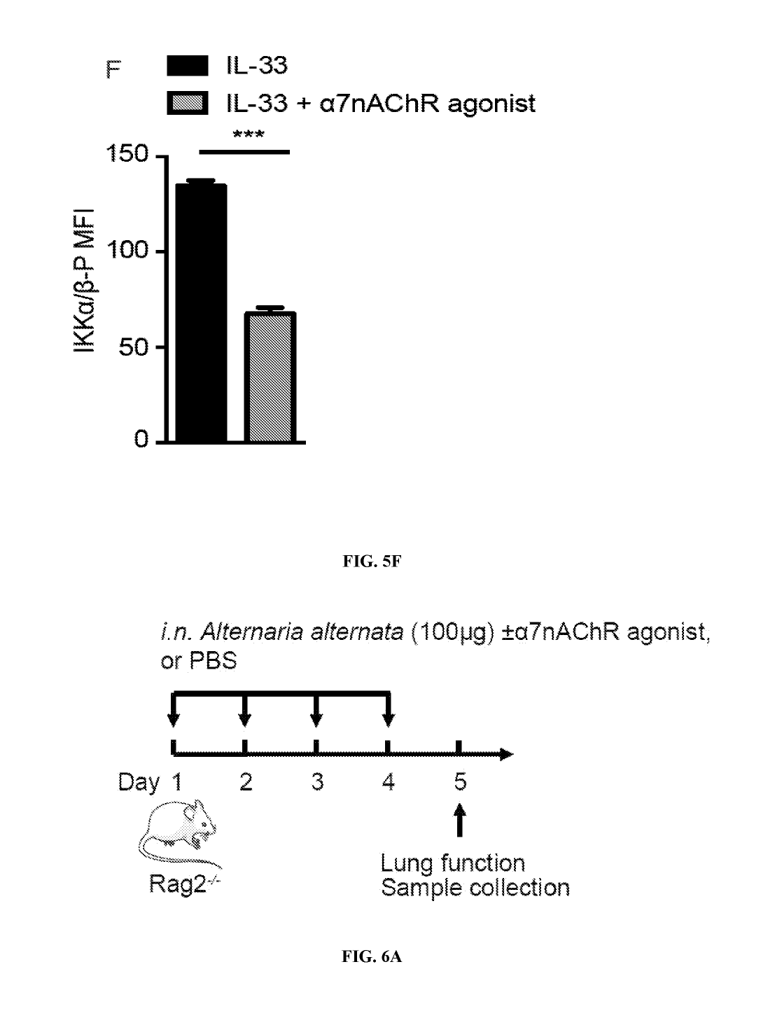

To characterize the mechanism enabling α7nAChR agonist to attenuate ILC2-mediated AHR, Applicants first investigated whether engagement of α7nAChR affects the development or maintenance of pulmonary ILC2s. However, Applicants observed a significantly decreased proliferation rate of pulmonary ILC2s upon α7nAChR agonist treatment by evaluating Ki-67 levels (FIG. 5A). Applicants also evaluated the expression of the transcription factor GATA binding protein-3 (GATA-3), which is essential for the development and maintenance of ILC2s³⁷⁻³⁹. Applicants found that α7nAChR agonist treatment significantly inhibits GATA-3 transcription in ILC2s (FIG. 5B). This decrease in GATA-3 expression could be involved in the repressed proliferation and function of ILC2s in response to α7nAChR agonist.

Next, to investigate the molecular mechanism of α7nAChR agonist-mediated anti-inflammatory effects, Applicants analyzed the gene expression profile of ILC2s with or without α7nAChR agonist treatment by NanoString technology as described in the methods. Evaluated genes were categorized and displayed in two panels; 1) genes mainly involved in the IL-33/IL-25 signaling pathway, and 2) genes associated with IL-2/IL-7 signaling pathway (FIG. 5C). As demonstrated in the IL-33/IL-25 signaling pathway, α7nAChR-mediated cholinergic activity inhibited expression of GATA-3 as well as STAT-6, NF-κB, IL-5, IL-9 and IL-13. Meanwhile, STAT-5a, STAT-5b, and IL-2 receptors in the IL-2/IL-7 signaling panel were relatively unaffected. Applicants also demonstrated that α7nAChR agonist did not activate factors associated with apoptosis, such as Casp3, Casp8, and Bcl-2. Applicants further confirmed by flow cytometry that α7nAChR agonist reduced NF-κB p65 expression in ILC2s (FIG. 5D). To better establish the underlying mechanisms, Applicants explored the signaling pathway upstream of NF-κB by measuring the activated form of IKKα/β, which is phosphorylated on serine residues 176 and 180. Consistent with our previous results shown in FIG. 5D, α7nAChR agonist reduced phosphorylated IKKα/β expression in ILC2s (FIGS. 5E-5F). NF-κB and STAT-6 are both critical for GATA-3 transcription. The inhibition of GATA-3 expression results in a down-regulation of IL-5, IL-9 and IL-13 secretion from ILC2s. Strikingly, these results demonstrate that α7nAChR stimulation likely reduces maintenance and development of ILC2s by inhibiting GATA-3 transcription, resulting in marked anti-inflammatory effects in the development of ILC2-mediated AHR and allergic inflammation.

α7nAChR Agonist Treatment Attenuates Allergen-Induced AHR

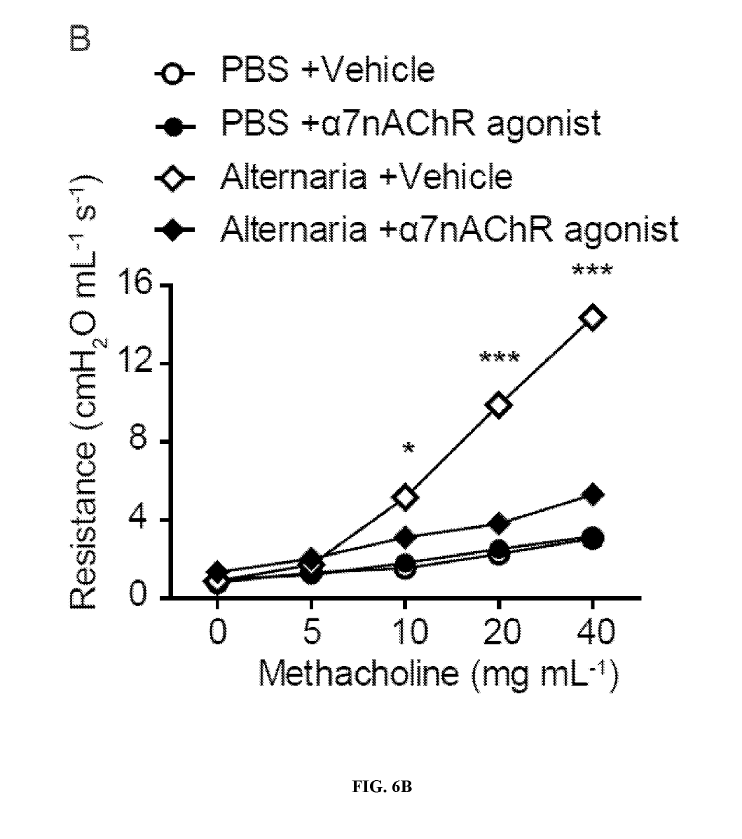

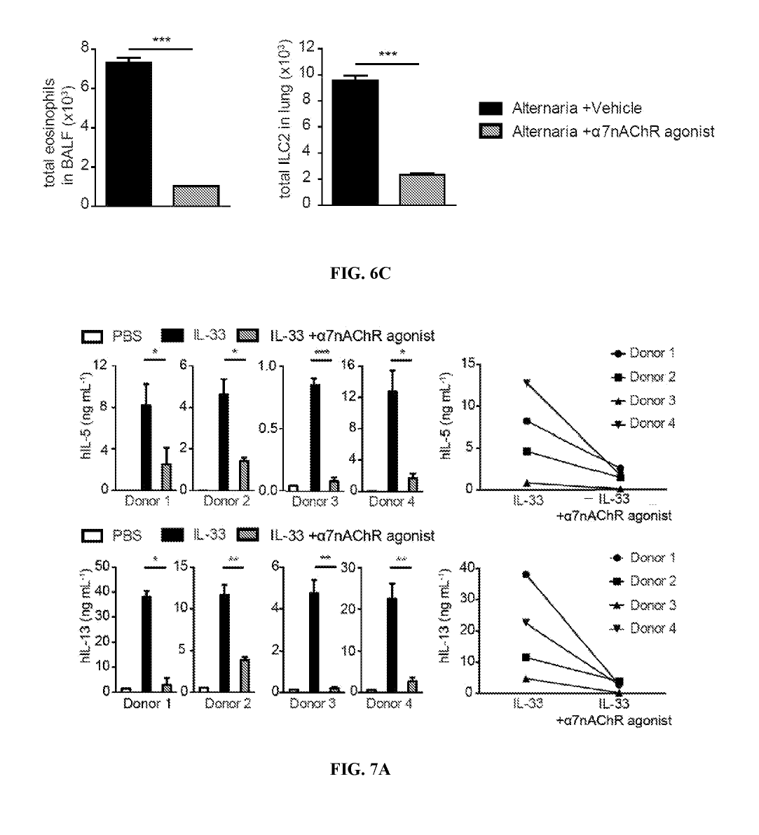

It has been previously reported that Alternaria alternata can induce AHR⁴⁰. Applicants further explored the anti-inflammatory effects of α7nAChR agonist in ILC2-mediated AHR with this clinically relevant allergen. Rag2^{-/-} mice were i.n. administered Alternaria alternata extract with or without α7nAChR agonist for four consecutive days (FIG. 6A). As expected, α7nAChR agonist treated mice did not develop AHR (FIG. 6B). Accordingly, the number of eosinophils in BALF and lung ILC2s were increased in Alternaria-treated mice. Meanwhile, α7nAChR agonist treatment abolished eosinophils and ILC2s in the lung (FIG. 6C). These results suggest α7nAChR agonist treatment can attenuate ILC2-mediated AHR in response to other allergens besides IL-33.

α7nAChR Agonist Prevents AHR in Humanized ILC2 Mice

To assess the effects of α7nAChR agonist in human ILC2s, peripheral blood mononuclear cells (PBMCs) were obtained from healthy donors and ILC2s were sorted on the basis of expression of CD45, CRTH2, CD127, and CD161, and the lack of human lineage markers (CD3, CD14, CD16, CD19, CD20, CD56, CD235a, CD1a, and CD123). Sorted ILC2s were cultured with α7nAChR agonist (10 μg mL⁻¹) in the presence of recombinant human (rh)-IL-2 (10 ng mL⁻¹), rh-IL-7 (20 ng mL⁻¹), and rh-IL-33 (20 ng mL⁻¹). As observed in our previous experiments (FIGS. 2A-2B), α7nAChR agonist significantly suppressed human ILC2 activation, by decreasing both IL-5 and IL-13 production (FIG. 7A).

Finally, to confirm the efficacy of α7nAChR agonist treatment in human ILC2-mediated AHR, sorted human ILC2s (hILC2s) were cultured in the presence of rh-IL-2 (20 ng mL⁻¹) and rh-IL-7 (20 ng mL⁻¹) for 48 hours. Subsequently, 2.5×10⁵ hILC2s were adoptively transferred to Rag2 GC double knockout mice, which lack T, B, NK cells and ILC2s. These humanized mice were given i.n. rh-IL33 (1 μg per mice) with or without α7nAChR agonists (125 □g mL⁻¹) for three consecutive days, and then AHR was evaluated (FIG. 7B). Consistent with the murine ILC2 experiments, treatment with α7nAChR agonist dampened ILC2-mediated AHR (FIG. 7C) and allergic inflammation (FIGS. 7D-7E) in humanized mice. Thus, α7nAChR mediates an anti-inflammatory signal in both murine and human ILC2s, suggesting α7nAChR activation with an agonist as a novel potential therapeutic strategy for regulating ILC2-mediated lung inflammatory diseases.

In the present study, using a leading compound with good selectivity and partial agonist activity toward α7nAChR, Applicants examined the involvement of α7nAChR in the pathogenesis of asthma to evaluate its potential as a therapeutic target. Applicants demonstrated, for the first time, that α7nAChR is expressed on ILC2s and up-regulated after engagement of the ST2 receptor by IL-33. By inhibiting GATA-3 transcription in ILC2, administration of α7nAChR agonist significantly attenuated the development and function of ILC2s, abolishing both AHR and allergic inflammation. Importantly, Applicants showed this agonist also ameliorated human ILC2-mediated AHR, indicating its potential as a novel therapeutic molecule for asthma patients.

α7nAChR is a neuronal subtype of nAChR composed of a homopentamer of α7 subunits that mediates pre and post-synaptic excitation. It is known that α7nAChR is located not only in the brain but also in the periphery, including immune cells such as effector T cells^{30,31}, B cells³², Tregs⁴¹, and macrophages¹⁵. Using an improved template based on crystal structures, Applicants performed a virtual screen for α7nAChR ligands and selected a leading compound, GTS-21, that has been previously characterized as a functional agonist of α7nAChR. Although GTS-21 had previously been shown to have anti-inflammatory effects^{42,43}, its role in ILC2-dependent AHR has not been examined to our knowledge. In line with previous studies, Applicants examined a newly identified lymphocyte population of ILC2s and showed that ILC2s expressed α7nAChR. Importantly, activated ILC2s upregulated α7nAChR expression more so than other immune cells, indicating that asthma patients having activated ILC2s might benefit greatly from a nicotinic agonist compound such as GTS-21.

Nicotine is just one of the over 4000 chemical constituents in tobacco smoke. Exposure to cigarette smoking causes impaired lung function and increases the risk of developing asthma^{44,45}. In asthma, patients who smoke have more symptoms and exacerbations than nonsmokers⁴⁶. They also have increased risks of hospitalization and mortality⁴⁷. On the other hand, epidemiological studies have indicated inverse correlations between smoking and incidence of allergic diseases. It is well known that the incidence of hypersensitive pneumonitides such as farmer's lung and bird fancier's lung is lower in the current smoker population than that in non-smokers⁴⁸⁻⁵¹. Furthermore, other clinical studies have observed that asthma incidence could be higher in former smoker populations compared to active smokers^{44,52}. In that longitudinal study, the observed increased asthma risks in former smokers were explained by the fact that, in some cases, asthma was self-reported or the supposition that people tend to quit smoking in response to respiratory symptoms. A cohort study also showed that development of allergic sensitization is negatively associated with sustained smoking⁵³. In accordance with these clinical findings, nicotine, a major constituent of cigarette smoke and a ligand for nAChR, has shown anti-inflammatory effects in various diseases^{31,54,55}. In asthma, nicotine attenuated HDM-induced allergic lung inflammation together with suppression of Th2 cytokines, although the underlying cellular mechanism remains unknown and AHR was not suppressed¹⁷. Moreover Kearley et al. recently demonstrated that cigarette smoke has suppressive effects on IL-5 and IL-13 production by ILC2s⁵⁶. In preclinical models of asthma, Applicants clearly demonstrated engagement of α7nAChR opposes the development of AHR and allergic inflammation through an ILC2-mediated mechanism. The differences in outcome likely resulted from the experimental design; our group administered a specific compound for α7nAChR together with rm-IL-33 or Alternaria alternata to mice, whilst Sopori et al. examined nicotine itself and HDM-induced AHR^{21,33}. In order to establish that our previous observations were dependent of α7nAChR expression on ILC2s, Applicants demonstrated that the α7nAChR agonist had no effect on IL-33-challenged mice that were deficient for α7nAChR on ILC2s. Collectively, our data can explain the cellular mechanisms behind the clinical anti-inflammatory observations.

Applicants demonstrated that α7nAChR activation altered ILC2 function in response to both exogenous IL-33 and Alternaria alternata. Administration of either IL-33 and IL-25 activates ILC2s to induce AHR independently of the adaptive immune system^{5,6,57}, and IL-33 was reported to be more potent than IL-25 in this regard⁵⁷. Alternaria alternata exposure also activates ILC2s and causes steroid-resistant AHR associated with elevated IL-33 in vivo 40. ILC2s rapidly produce huge amounts of IL5 and IL-13 in response to stimuli. A recent study showed that ILC2-derived IL-13 is capable of inducing differentiation among Th2 cells³⁵. Based on these reports, regulating ILC2s could potentially control allergic inflammation arising from not only the innate, but also adaptive, immune pathway. By targeting ILC2 function, α7nAChR agonist treatments could prove remarkably therapeutic for various allergic diseases.

To explore the mechanisms underlying the anti-inflammatory effects of the α7nAChR agonist, Applicants initially demonstrated the engagement of α7nAChR caused reduced number of ILC2s in the lung, suggesting involvement of the cholinergic signal in cell fate and maintenance. Applicants next found that α7nAChR agonist significantly suppressed Ki67 expression, a cellular marker for proliferation in ILC2s. In contrast, expression of anti-apoptotic factor Bcl-2 on ILC2s was unaffected by α7nAChR stimulation. In accordance with these in vivo data, viability of ILC2s in culture and results from the NanoString assay were comparable. These results suggest that cholinergic signal transmission regulates proliferation, but not cell death, in ILC2s. Our findings are consistent with the cholinergic underpinnings of anti-inflammatory mechanisms in other diseases of inflammation, such as autoimmune arthritis and experimental autoimmune encephalomyelitis^{31,54,55}.

On one hand, Dowling et al. also described that nicotine could inhibit the NF-κB pathway in an α7nAChR-dependent manner⁵⁸. Applicants demonstrated by cytometry that in response to the agonist, the expression of the activated NF-κB p65 subunit was reduced. Similarly Applicants also observed a decrease in the expression at the mRNA level of NF-κB1. However, with the Nanostring Technology Applicants also noticed that RelA expression was unaffected. This discrepancy could be explained by the fact that NF-κB is mainly regulated at the post-translational level by phosphorylation. Phosphorylation of the NF-κB p65 subunit on certain residues plays a key role in regulating NF-κB activation and function. Applicants also validate the results by assessing IKKα/β, upstream of NF-κB and observed a significant reduction in IKKα/β after agonist treatment.

On the other hand, in cancer immunity, nicotine administration enhances tumor growth by promoting cell proliferation and suppressing apoptosis⁵⁹⁻⁶². Strikingly, these results suggest that cholinergic signal is involved in the pathogenesis of various diseases, with a distinct role in each disease.

In addition to attenuated proliferation, α7nAChR agonist also inhibited GATA-3 expression in ILC2s. GATA-3, a double zinc-finger transcription factor, is required for the development of Th2 cells and important for the production of IL-5 and IL-13⁶³⁻⁶⁵. Recently, GATA-3 was also reported to be essential for differentiation and maintenance of ILC2s, and their production of IL-5 and IL-13³⁷⁻³⁹. Moreover, higher expression of GATA-3 is associated with an increase in ILC2-derived IL-13⁶⁶. Collectively, these results indicate an underlying mechanism for the anti-inflammatory role of α7nAChR agonist in asthma: modulating GATA-3 expression and proliferation in ILC2s, which subsequently attenuates Th2 cytokine production from ILC2s, preventing the development of AHR and allergic inflammation.

Applicants further investigated whether this cholinergic anti-inflammatory effect was relevant in a humanized mice model, in which human ILC2s were adoptively transferred to Rag^{-/-} IL2rg^{-/-} mice and administered IL-33 to induce AHR. This unique system allows one to directly evaluate human ILC2-mediated AHR³⁶. In accordance with the effects seen in murine ILC2s, α7nAChR agonist significantly suppressed human ILC2 function and dampened human ILC2-mediated AHR. Taken in their entirety, our results suggest that enhancing cholinergic signal might be an effective therapeutic strategy for patients with ILC2-mediated asthma.

In conclusion, this is the first report to reveal that the engagement of α7nAChR on ILC2s suppresses AHR. Therefore, our results suggest a protective role of cholinergic signaling in the pathogenesis of asthma, and present α7nAChR agonists as novel therapeutic candidates for controlling ILC2-mediated inflammatory lung diseases.

### Mice

Female BALB/cByJ, RAG2 deficient (C.B6(Cg)-Rag2^{tm1.1Cgn}/J) mice, RAG2 GC deficient (C; 129S4-Rag2^{tm1.1Flv} IL2rg^{tm1.1Flv}/J) mice and α7nAChR deficient (B6.129S7-Chrna7^{tm1Bay}/J) (6 to 8 weeks old) were purchased from Jackson Laboratory (Bar Harbor, Me.). Rag2 deficient and Rag2 GC deficient mice were bred in our facility at the Keck School of Medicine, University of Southern California (USC). Animal studies were approved by the USC Institutional Animal Care and Use Committee and conducted in accordance with the USC Department of Animal Resources' guidelines. All human studies were approved by USC institutional review board and conducted according to the principles of the Declaration of Helsinki. Participants gave written informed consent to prior to their inclusion in the study, and were identified by number.

### Crystallization and Structure Determination of Agonist

The structure of the agonist 4-OH-GTS-21 was docked into the ligand site of the crystal structure of the α7nAChR Chimera²¹ using our previously published computation docking procedures²². The figure is made by PyMOL (The PyMOL Molecular Graphics System, v 1.8, Schrodinger, LLC).

### Measurement of Airway Hyperreactivity

Mice were intranasally (i.n.) administered carrier-free recombinant mouse IL-33 (BioLegend, San Diego Calif., 0.5 □g per mouse in 50 □L) with or without 125 μL of α7nAChR agonist (kindly provided by Lin Chen) on three consecutive days. Mice were intranasally (i.n.) administered carrier-free recombinant mouse IL-25 (BioLegend, San Diego Calif., 5 □g per mouse in 50 □L) with or without 125 μL of α7nAChR agonist on three consecutive days. For Alternaria alternata experiments, mice were i.n. administered Alternaria alternata (Greer Labs, Lenoir, N.C., 100 μg per mouse in 50 μL) with or without 125 □g α7nAChR agonist for four consecutive days. One day after the last challenge, mice were anesthetized using i.p. injection of ketamine (10 mg mL⁻¹) and xylazine (1 mg mL⁻¹). Measurements of airway resistance and dynamic compliance were conducted with the Fine Pointe RC System (Buxco Research Systems, Wilmington, N.C.), in which mice were mechanically ventilated using a modified version as previously described^{36,67}. Mice were sequentially challenged with aerosolized PBS (baseline), followed by increasing doses of methacholine. Maximum lung resistance (R_{L}) and minimum compliance (C_{dyn}) values were recorded during a three-minute period after each methacholine challenge.

### Collection of BAL Cells and Lung Histology

After measurements of AHR, the trachea was canulated and the lungs lavaged three times with 1 mL ice cold PBS to collect BALF cells as previously described⁶⁸. BALF cells were stained with allophycocyanin (APC)-labeled anti-Ly-6G/Ly-6C (clone RB6-8C5, BioLegend, San Diego, Calif.), Alexa Fluor-labeled anti-CD19 (clone 6D5, BioLegend), phycoerythrin (PE)-labeled anti-Singlec-F (clone E50-2440, BD Pharmingen, San Diego, Calif.), and PE-Cy (PE-Cy7) labeled anti-CD45 (clone 30-F11, BioLegend), peridinin-chlorophyll-protein complex-Cy5.5 (PerCP-Cy5.5) labeled anti-CD3e (clone145-2C11, eBioscience, San Diego), and eFluor-450 labeled anti-CD11b (clone M1/70, eBioscience), and APC-Cy7 labeled anti-CD11c (clone N418, BioLegend). Transcardinal perfusion of the lungs with cold PBS was then performed to remove red blood cells and the lungs fixed and harvested for histology with 4% paraformaldehyde in PBS. After fixation, the lungs were embedded in paraffin, cut into 4 □m sections, a

[symbol]

### Flow Cytometry

Biotinylated anti-mouse lineage (CD3e (145-2C11), CD45R (RA3-3B2), Gr-1 (RB6-8C5), CD11c (N418), CD11b (M1/70), Ter119 (TER-119), NK1.1 (PK136), TCR-β (H57-597), TCR-γδ (GL3), and FcεRIα (MAR-1), Streptavidin-FITC, FITC anti-mouse CD3 (145-2C11), BV510 anti-mouse CD4 (RM4-5), APC-Cy7 anti-mouse CD25 (PC61), PE anti-mouse CD25 (PC61), BV421 anti-mouse CD25 (PC61), BV510 anti-mouse CD90.2 (53-2.1), APC anti-mouse CD127 (A7R34), PE-Cy7 anti-mouse CD127 (A7R34), FITC anti-mouse CD45 (30F-11), PE/Cy7 anti-mouse CD45 (30-F11), PE anti-mouse IL-5 (TRFK5), BV421 anti-mouse GATA3 (16E10A23), 7-AAD Viability staining solution, purchased from BioLegend (San Diego, Calif.). PE anti-mouse ST2 (IL-33R, RMST2-2), PerCP-eFluor710 anti-Mouse ST2 (RMST2-2), Streptavidin APC-eFluor780, Alexa Fluor647 labeled anti-IL-4 (clone 11B11), eFluor 450 anti-mouse CD45 (30F-11), eFluor450 anti-CD44 (clone IM7), eFluor-660 anti-mouse Ki-67 (SollA15), PE/Cy7 anti-mouse IL-13 (eBio13A), anti-mouse BCL-2 (10C4), were purchased from eBioscience. Alexa Fluor 488 anti-mouse p65-NF-κB (532301) was purchased from R&D Systems (dilution 1:50). Alexa Fluor 647 conjugated α-bungarotoxin (Invitrogen, San Diego, Calif.). PE anti-mouse Phospho-IKKα/β (Ser 176/180) (16A6) was purchased from Cell Signaling Technology (dilution 1:50). PE anti-rat α7nAChR (319) was purchased from Santa Cruz Biotechnology (dilution 1:50). This antibody has been used to identify this receptor previously in several studies⁶⁹. Lineage marker antibodies were used at the dilution of 1:400, unless mentioned otherwise other antibodies were used at the dilution of 1:200.

BD Cytofix™Fixation Buffer and BD Phosflow™ Perm Buffer III were purchased from BD Biosciences (San Jose, Calif.). Flow cytometry was carried out on the FACSCanto II and FACSARIA III (BD Biosciences) and the data were analyzed with FlowJo version 8.6 software (TreeStar, Ashland, Oreg.).

### Identification of Mouse ILC2s

Lung ILC2s were defined as lack of classical lineage markers (CD3e, CD45R, Gr-1, CD11c, CD11b, Ter119, NK1.1, TCR-β, TCR-γδ and FcεRIα) and CD25⁺, CD45⁺, CD90.2⁺, and ST2⁺ populations.

### Intracellular Staining

Intracellular staining was performed using BD Cytofix/Cytoperm kit (BD Bioscience, San Jose, Calif.) according to the manufacturer's instructions. For analysis of GATA3 and Ki-67 expression, freshly isolated cells were fixed and permeabilized using Fixation/Permeabilization buffer kit (eBioscience) according to the manufacturer's instructions and as previously described⁷⁰.

### Humanized Mice and Purification of Human ILC2

For human peripheral ILC2s, peripheral blood mononuclear cells (PBMCs) were first isolated from human fresh blood by diluting the blood 1:1 in PBS, adding to SepMate™-50 separation tubes (STEMCELL Technologies Inc, Vancouver, Canada) prefilled with 15-mL Lymphoprep™ each (Axis-Shield, Oslo, Norway), and centrifuging at 1200 g for 15 minutes. Human PBMCs were then washed in PBS and stained with antibodies against human lineage markers (CD3, CD14, CD16, CD19, CD20, CD56, CD235a, CD1a, CD123), CRTH2, CD161, CD127, and CD45. Thereafter, human ILC2s were defined as CD45+ lineage− CRTH2+ CD127+ CD161+ and purified by flow cytometry using BD FACS ARIA III (BD Biosciences, San Jose, Calif.) with a purity of >95%. Purified human ILC2s were cultured with rh-IL2 (20 ng mL⁻¹) and rh-IL-7 (20 ng mL⁻¹) for 48 hours, then adoptively transferred to Rag2 GC double knockout mice (2.5×10⁵ cells per mouse) followed by i.n. administration of recombinant human IL-33 (0.5 μg per mouse) with or without α7nAChR agonist on day 1-3. On day 4, lung function was measured and BAL was performed and analyzed.

### In Vitro Stimulation of ILC2s

Murine ILC2s were isolated from BALB/cByJ mice to >95% purity using the FACSARIA III cell sorter. Isolated murine ILC2s (5.0×10⁴ per mL) were stimulated with rm-IL-33 (20 ng mL⁻¹), rm-IL-2 (10 ng mL⁻¹), and rm-IL-7 (10 ng mL⁻¹) in the presence or absence of increasing doses of α7nAChR agonist (2.5 μg mL⁻¹, 10 μg mL⁻¹, 50 μg mL⁻¹) for 1 or 2 days. Isolated murine ILC2s (5.0×10⁴ per mL) were stimulated with rm-IL-25 (10 ng mL⁻¹) in the presence or absence of α7nAChR agonist (10 μg mL⁻¹) for 24 hours. For human ILC2s in vitro culture, isolated human ILC2s (1.0×10⁵ per mL) were cultured with rh-IL-33 (20 ng mL⁻¹), rh-IL-2 (10 ng mL⁻¹), and rh-IL-7 (20 ng mL⁻¹) in the presence of α7nAChR (10 μg mL⁻¹). Levels of cytokines were measured by ELISA (eBioscience), according to the manufacturer's instructions.

### Reverse Transcription-Polymerase Chain Reaction (RT-PCR)

The expression of α7 and α4 genes was quantitated at the mRNA levels by quantitative real-time PCR (qPCR). Total RNA was extracted with the RNeasy Mini Kit (Qiagen, Valencia, Calif.), and α7nChR (forward, 5′-CTCTGACTGTCTTCATGCTGCT-3′ and reverse, 5′-ATCATGGTGCTGGCGAAG-3′) and α4nAChR (forward, 5′-CGTCCAGTACATTGCAGACC-3′ and reverse, 5′-ATGACCATGGCCACGTATTT-3′) genes were quantified at the mRNA level by quantitative real-time PCR (qPCR). Total RNA was extracted with the RNeasy Mini Kit (Qiagen, Valencia, Calif.), and the α7 and α4 mRNA expressions were measured using the TaqMan gene expression assay at the Applied Biosystems 7500 system (Applied Biosystems, Carlsbad, Calif.) in accordance with the manufacturer's protocol.

Gene Expression Analysis with NanoString nCounter Technology

The difference in the abundance of scripts between ILC2s purified from rm-IL33 administered mice with or without α7nAChR agonist were analyzed with NanoString nCounter technology. Heat plots were generated with R statistical software.

### Statistical Analysis

A student t test was used for comparisons between each group. P values of less than 0.05 were considered significant. All data are expressed as the mean±SEM.

### GTS-21 Acts Selectively

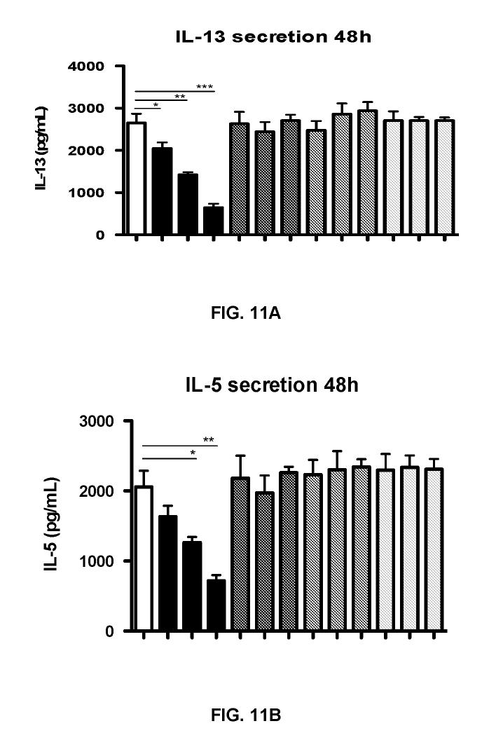

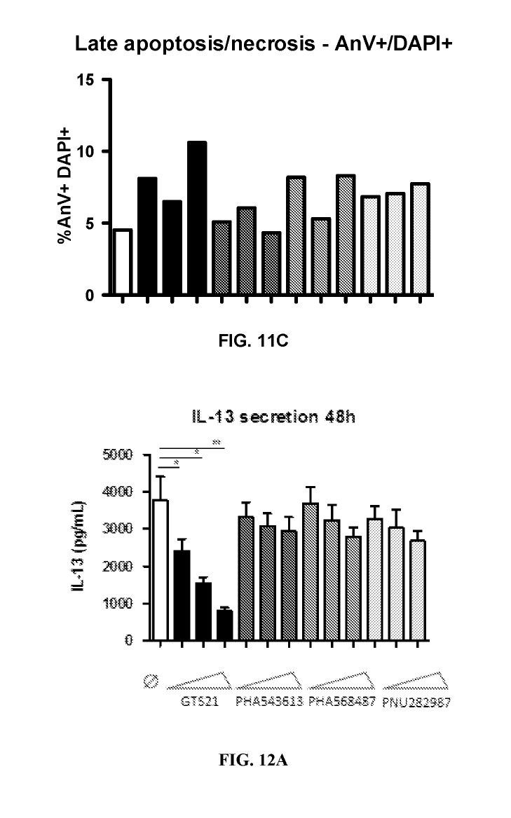

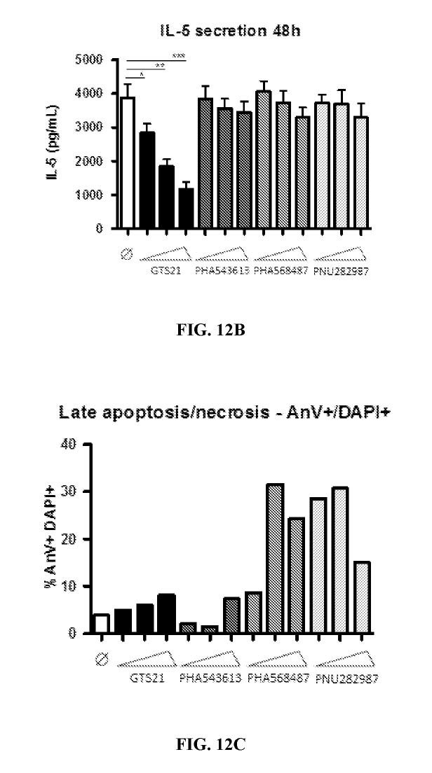

As shown in FIGS. 11A-11C and 12A-12C, other nicotine agonists did not provide the same therapeutic benefit as GTS-21. FIGS. 11A-11C show the results of an experiment wherein BALB/c mice were intranasally challenged with 0.5 μg rmIL-33 on days 1-3. On day 4 lung ILC2s were isolated and restimulated with 10 ng/mL rmIL-2 and rmIL-7 with or without increased concentrations of the indicated nicotine agonists for 48 h. The levels of (FIG. 11A) IL-13 and (FIG. 11B) IL-5 in the culture supernatants were measured by ELISA. (FIG. 11C) The viability of cultured ILC2s was measured by flow cytometry by staining cells with Annexin V (AnV) and DAPI. Values are presented as mean±/−SEM with n=6, * p<0.05, ** p<0.01, *** p<0.001. Similarly as shown in FIGS. 12A-12C, BALB/c mice were intranasally challenged with 0.5 μg rmIL-33 on days 1-3. On day 4 lung ILC2s were isolated and restimulated with 10 ng/mL rmIL-2 and rmIL-7 with or without increased concentrations of the indicated nicotine agonists for 48 h. The levels of (12A) IL-13 and (12B) IL-5 in the culture supernatants were measured by ELISA. (12C) The viability of cultured ILC2s was measured by flow cytometry by staining cells with Annexin V (AnV) and DAPI. Values are presented as mean±/−SEM with n=6, * p<0.05, ** p<0.01, *** p<0.001.

### EQUIVALENTS

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The invention has been described broadly and generically herein. Each of the narrower species and subgeneric groupings falling within the generic disclosure also form part of the invention. In addition, where features or aspects of the invention are described in terms of Markush groups, those skilled in the art will recognize that the invention is also thereby described in terms of any individual member or subgroup of members of the Markush group.

### REFERENCES

1 Kim, H. Y, DeKruyff, R. H. & Umetsu, D. T. The many paths to asthma: phenotype shaped by innate and adaptive immunity. Nat Immunol 11, 577-584, doi:10.1038/ni.1892 (2010).
2 Lambrecht, B. N. & Hammad, H. The airway epithelium in asthma. Nat Med 18, 684-692, doi:10.1038/nm.2737 (2012).
3 Johnston, R. A. et al. Allergic airway responses in obese mice. Am J Respir Crit Care Med 176, 650-658, doi:10.1164/rccm.200702-3230C (2007).
4 Kim, E. Y et al. Persistent activation of an innate immune response translates respiratory viral infection into chronic lung disease. Nat Med 14, 633-640, doi:10.1038/nm1770 (2008).
5 Chang, Y J. et al. Innate lymphoid cells mediate influenza-induced airway hyperreactivity independently of adaptive immunity. Nat Immunol 12, 631-638, doi:10.1038/ni.2045 (2011).
6 Yu, S., Kim, H. Y, Chang, Y J., DeKruyff, R. H. & Umetsu, D. T. Innate lymphoid cells and asthma. The Journal of allergy and clinical immunology 133, 943-950; quiz 951, doi:10.1016/j.jaci.2014.02.015 (2014).
7 Artis, D. & Spits, H. The biology of innate lymphoid cells. Nature 517, 293-301, doi:10.1038/nature14189 (2015).
8 Bartemes, K. R. et al. IL-33-responsive lineage− CD25+ CD44(hi) lymphoid cells mediate innate type 2 immunity and allergic inflammation in the lungs. J Immunol 188, 1503-1513, doi:10.4049/jimmunol.1102832 (2012).
9 Halim, T. Y, Krauss, R. H., Sun, A. C. & Takei, F. Lung natural helper cells are a critical source of Th2 cell-type cytokines in protease allergen-induced airway inflammation. Immunity 36, 451-463, doi:10.1016/j.immuni.2011.12.020 (2012).
10 Molofsky, A. B. et al. Interleukin-33 and Interferon-gamma Counter-Regulate Group 2 Innate Lymphoid Cell Activation during Immune Perturbation. Immunity 43, 161-174, doi:10.1016/j.immuni.2015.05.019 (2015).
11 Mirchandani, A. S. et al. Type 2 innate lymphoid cells drive CD4+ Th2 cell responses. J Immunol 192, 2442-2448, doi:10.4049/jimmunol.1300974 (2014).
12 Neill, D. R. et al. Nuocytes represent a new innate effector leukocyte that mediates type-2 immunity. Nature 464, 1367-1370, doi:10.1038/nature08900 (2010).
13 Martin, L. F., Kem, W. R. & Freedman, R. Alpha-7 nicotinic receptor agonists: potential new candidates for the treatment of schizophrenia. Psychopharmacology (Berl) 174, 54-64, doi:10.1007/s00213-003-1750-1 (2004).
14 D'Andrea, M. R. & Nagele, R. G. Targeting the alpha 7 nicotinic acetylcholine receptor to reduce amyloid accumulation in Alzheimer's disease pyramidal neurons. Curr Pharm Des 12, 677-684 (2006).
15 Wang, H. et al. Nicotinic acetylcholine receptor alpha7 subunit is an essential regulator of inflammation. Nature 421, 384-388, doi:10.1038/nature01339 (2003).
16 Borovikova, L. V. et al. Vagus nerve stimulation attenuates the systemic inflammatory response to endotoxin. Nature 405, 458-462, doi:10.1038/35013070 (2000).
17 Mishra, N. C. et al. Nicotine primarily suppresses lung Th2 but not goblet cell and muscle cell responses to allergens. J Immunol 180, 7655-7663 (2008).
18 Mishra, N. C. et al. Nicotine inhibits Fc epsilon RI-induced cysteinyl leukotrienes and cytokine production without affecting mast cell degranulation through alpha 7/alpha 9/alpha 10-nicotinic receptors. J Immunol 185, 588-596, doi:10.4049/jimmunol.0902227 (2010).
19 Hurst, R., Rollema, H. & Bertrand, D. Nicotinic acetylcholine receptors: from basic science to therapeutics. Pharmacology & therapeutics 137, 22-54, doi:10.1016/j.pharmthera.2012.08.012 (2013).
20 Mazurov, A. A., Speake, J. D. & Yohannes, D. Discovery and development of alpha7 nicotinic acetylcholine receptor modulators. Journal of medicinal chemistry 54, 7943-7961, doi:10.1021/jm2007672 (2011).
21 Li, S. X. et al. Ligand-binding domain of an alpha7-nicotinic receptor chimera and its complex with agonist. Nat Neurosci 14, 1253-1259, doi:10.1038/nn.2908 (2011).
22 Dey, R. & Chen, L. In search of allosteric modulators of a7-nAChR by solvent density guided virtual screening. J Biomol Struct Dyn 28, 695-715, doi:10.1080/07391102.2011. Ser. No. 10/508,600 (2011).
23 Akdemir, A. et al. Identification of novel alpha7 nicotinic receptor ligands by in silico screening against the crystal structure of a chimeric alpha7 receptor ligand binding domain. Bioorganic & medicinal chemistry 20, 5992-6002, doi:10.1016/j.bmc.2012.06.054 (2012).
24 van Haaren, F., Anderson, K. G., Haworth, S. C. & Kem, W. R. GTS-21, a mixed nicotinic receptor agonist/antagonist, does not affect the nicotine cue. Pharmacol Biochem Behav 64, 439-444 (1999).
25 Kem, W. R. et al. Hydroxy metabolites of the Alzheimer's drug candidate 3-[(2,4-dimethoxy)benzylidene]-anabaseine dihydrochloride (GTS-21): their molecular properties, interactions with brain nicotinic receptors, and brain penetration. Molecular pharmacology 65, 56-67, doi:10.1124/mol.65.1.56 (2004).
26 Huang, S. et al. Complex between alpha-bungarotoxin and an alpha7 nicotinic receptor ligand-binding domain chimaera. Biochem J 454, 303-310, doi:10.1042/bj20130636 (2013).
27 Stevens, K. E. et al. Continuous administration of a selective alpha7 nicotinic partial agonist, DMXBA, improves sensory inhibition without causing tachyphylaxis or receptor upregulation in DBA/2 mice. Brain Res 1352, 140-146, doi:10.1016/j.brainres.2010.06.063 (2010).
28 Kem, W. R. Alzheimer's drug design based upon an invertebrate toxin (anabaseine) which is a potent nicotinic receptor agonist. Invert Neurosci 3, 251-259 (1997).
29 Hibbs, R. E. et al. Structural determinants for interaction of partial agonists with acetylcholine binding protein and neuronal alpha7 nicotinic acetylcholine receptor. Emboj 28, 3040-3051, doi:10.1038/emboj.2009.227 (2009).
30 Nizri, E., Hamra-Amitay, Y, Sicsic, C., Lavon, I. & Brenner, T. Anti-inflammatory properties of cholinergic up-regulation: A new role for acetylcholinesterase inhibitors. Neuropharmacology 50, 540-547, doi:10.1016/j.neuropharm.2005.10.013 (2006).
31 Nizri, E. et al. Activation of the cholinergic anti-inflammatory system by nicotine attenuates neuroinflammation via suppression of Th1 and Th17 responses. J Immunol 183, 6681-6688, doi:10.4049/jimmunol.0902212 (2009).
32 Skok, M. V, Grailhe, R., Agenes, F. & Changeux, J. P. The role of nicotinic receptors in B-lymphocyte development and activation. Life Sci 80, 2334-2336, doi:10.1016/j.lfs.2007.02.005 (2007).
33 Dellisanti, C. D., Yao, Y, Stroud, J. C., Wang, Z. Z. & Chen, L. Crystal structure of the extracellular domain of nAChR alpha1 bound to alpha-bungarotoxin at 1.94 A resolution. Nat Neurosci 10, 953-962, doi:10.1038/nn 1942 (2007).
34 Walker, J. A., Barlow, J. L. & McKenzie, A. N. Innate lymphoid cells—how did we miss them? Nat Rev Immunol 13, 75-87, doi:10.1038/nri3349 (2013).
35 Halim, T. Y et al. Group 2 innate lymphoid cells are critical for the initiation of adaptive T helper 2 cell-mediated allergic lung inflammation. Immunity 40, 425-435, doi:10.1016/j.immuni.2014.01.011 (2014).
36 Maazi, H. et al. ICOS:ICOS-ligand interaction is required for type 2 innate lymphoid cell function, homeostasis, and induction of airway hyperreactivity. Immunity 42, 538-551, doi:10.1016/j.immuni.2015.02.007 (2015).
37 Hoyler, T. et al. The transcription factor GATA-3 controls cell fate and maintenance of type 2 innate lymphoid cells. Immunity 37, 634-648, doi:10.1016/j.immuni.2012.06.020 (2012).
38 Klein Wolterink, R. G. et al. Essential, dose-dependent role for the transcription factor Gata3 in the development of IL-5+ and IL-13+ type 2 innate lymphoid cells. Proc Natl Acad Sci USA 110, 10240-10245, doi:10.1073/pnas.1217158110 (2013).
39 Mjosberg, J. et al. The transcription factor GATA3 is essential for the function of human type 2 innate lymphoid cells. Immunity 37, 649-659, doi:10.1016/j.immuni.2012.08.015 (2012).
40 Castanhinha, S. et al. Pediatric severe asthma with fungal sensitization is mediated by steroid-resistant IL-33. The Journal of allergy and clinical immunology, doi:10.1016/j.jaci.2015.01.016 (2015).
41 Wang, D. W. et al. Stimulation of alpha7 nicotinic acetylcholine receptor by nicotine increases suppressive capacity of naturally occurring CD4+CD25+ regulatory T cells in mice in vitro. The Journal of pharmacology and experimental therapeutics 335, 553-561, doi:10.1124/jpet.110.169961 (2010).
42 Kox, M. et al. Effects of the alpha7 nicotinic acetylcholine receptor agonist GTS-21 on the innate immune response in humans. Shock 36, 5-11, doi:10.1097/SHK.0b013e3182168d56 (2011).
43 Bruchfeld, A. et al. Whole blood cytokine attenuation by cholinergic agonists ex vivo and relationship to vagus nerve activity in rheumatoid arthritis. J Intern Med 268, 94-101, doi:10.1111/j.1365-2796.2010.02226.x (2010).
44 Godtfredsen, N. S., Lange, P., Prescott, E., Osler, M. & Vestbo, J. Changes in smoking habits and risk of asthma: a longitudinal population based study. Eur Respir J 18, 549-554 (2001).
45 Pietinalho, A., Pelkonen, A. & Rytila, P. Linkage between smoking and asthma. Allergy 64, 1722-1727, doi:10.1111/j.1398-9995.2009.02208.x (2009).
46 Siroux, V, Pin, I., Oryszczyn, M. P., Le Moual, N. & Kauffmann, F. Relationships of active smoking to asthma and asthma severity in the EGEA study. Epidemiological study on the Genetics and Environment of Asthma. Eur Respir J 15, 470-477 (2000).
47 Marquette, C. H. et al. Long-term prognosis of near-fatal asthma. A 6-year follow-up study of 145 asthmatic patients who underwent mechanical ventilation for a near-fatal attack of asthma. Am Rev Respir Dis 146, 76-81, doi:10.1164/ajrccm/146.1.76 (1992).
48 Warren, C. P. Extrinsic allergic alveolitis: a disease commoner in non-smokers. Thorax 32, 567-569 (1977).
49 McSharry, C., Banham, S. W. & Boyd, G. Effect of cigarette smoking on the antibody response to inhaled antigens and the prevalence of extrinsic allergic alveolitis among pigeon breeders. Clin Allergy 15, 487-494 (1985).
50 Anderson, K., Morrison, S. M., Bourke, S. & Boyd, G. Effect of cigarette smoking on the specific antibody response in pigeon fanciers. Thorax 43, 798-800 (1988).
51 Carrillo, T., Rodriguez de Castro, F., Cuevas, M., Diaz, F. & Cabrera, P. Effect of cigarette smoking on the humoral immune response in pigeon fanciers. Allergy 46, 241-244 (1991).
52 Langhammer, A., Johnsen, R., Holmen, J., Gulsvik, A. & Bjermer, L. Cigarette smoking gives more respiratory symptoms among women than among men. The Nord-Trondelag Health Study (HUNT). Journal of epidemiology and community health 54, 917-922 (2000).
53 Linneberg, A. et al. Smoking and the development of allergic sensitization to aeroallergens in adults: a prospective population-based study. The Copenhagen Allergy Study. Allergy 56, 328-332 (2001).
54 Yu, H., Yang, Y H., Rajaiah, R. & Moudgil, K. D. Nicotine-induced differential modulation of autoimmune arthritis in the Lewis rat involves changes in interleukin-17 and anti-cyclic citrullinated peptide antibodies. Arthritis Rheum 63, 981-991, doi:10.1002/art.30219 (2011).
55 Shi, F. D. et al. Nicotinic attenuation of central nervous system inflammation and autoimmunity. J Immunol 182, 1730-1739 (2009).
56 Kearley, J. et al. Cigarette smoke silences innate lymphoid cell function and facilitates an exacerbated type I interleukin-33-dependent response to infection. Immunity 42, 566-579, doi:10.1016/j.immuni.2015.02.011 (2015).
57 Barlow, J. L. et al. IL-33 is more potent than IL-25 in provoking IL-13-producing nuocytes (type 2 innate lymphoid cells) and airway contraction. The Journal of allergy and clinical immunology 132, 933-941, doi:10.1016/j.jaci.2013.05.012 (2013).
58 Dowling, O., Rochelson, B., Way, K., Al-Abed, Y & Metz, C. N. Nicotine inhibits cytokine production by placenta cells via NFkappaB: potential role in pregnancy-induced hypertension. Molecular medicine (Cambridge, Mass.) 13, 576-583, doi:10.2119/2007-00067.Dowling (2007).
59 Cesario, A., Russo, P., Nastrucci, C. & Granone, P. Is alpha7-nAChR a possible target for lung cancer and malignant pleural mesothelioma treatment? Curr Drug Targets 13, 688-694 (2012).
60 West, K. A. et al. Rapid Akt activation by nicotine and a tobacco carcinogen modulates the phenotype of normal human airway epithelial cells. J Clin Invest 111, 81-90, doi:10.1172/jci16147 (2003).
61 Dasgupta, P. et al. Nicotine inhibits apoptosis induced by chemotherapeutic drugs by up-regulating XIAP and survivin. Proc Natl Acad Sci USA 103, 6332-6337, doi:10.1073/pnas.0509313103 (2006).
62 Schaal, C. & Chellappan, S. P. Nicotine-mediated cell proliferation and tumor progression in smoking-related cancers. Mol Cancer Res 12, 14-23, doi:10.1158/1541-7786.mcr-13-0541 (2014).
63 Zheng, W. & Flavell, R. A. The transcription factor GATA-3 is necessary and sufficient for Th2 cytokine gene expression in CD4 T cells. Cell 89, 587-596 (1997).
64 Ouyang, W. et al. Inhibition of Th1 development mediated by GATA-3 through an IL-4-independent mechanism. Immunity 9, 745-755 (1998).
65 Ansel, K. M., Djuretic, I., Tanasa, B. & Rao, A. Regulation of Th2 differentiation and 114 locus accessibility. Annu Rev Immunol 24, 607-656, doi:10.1146/annurev.immunol.23.021704.115821 (2006).
66 Liang, H. E. et al. Divergent expression patterns of IL-4 and IL-13 define unique functions in allergic immunity. Nat Immunol 13, 58-66, doi:10.1038/ni.2182 (2012).
67 Kerzerho, J. et al. Programmed cell death ligand 2 regulates TH9 differentiation and induction of chronic airway hyperreactivity. The Journal of allergy and clinical immunology 131, 1048-1057, 1057.e1041-1042, doi:10.1016/j.jaci.2012.09.027 (2013).
68 Maazi, H. et al. Lack of PD-L1 expression by iNKT cells improves the course of influenza A infection. PloS one 8, e59599, doi:10.1371/journal.pone.0059599 (2013).
69 Aizawa, K. et al. Development of ferret as a human lung cancer model by injecting 4-(Nmethyl-N-nitrosamino)-1-(3-pyridyl)-1-butanone (NNK). Lung cancer (Amsterdam, Netherlands) 82, 390-396 (2013).
70 Suzuki, Y et al. Lack of autophagy induces steroid-resistant airway inflammation. The Journal of allergy and clinical immunology 137, 1382-1389.e1389, doi:10.1016/j.jaci.2015.09.033 (2016).

1. The method of any one or more of
a. treating and/or ameliorating an inflammatory condition,
b. reducing ILC2 effector function;
c repressing IC2-dependent AHR;
d. decreasing expression of ILC2 transcription factor GATA-3;
e. decreasing expression of ILC2 inflammatory modulator NF-κB;
f. reducing phosphorylation of kinase IKKα/β;
g. reducing ILC2-mediated cytokine production; or
h. treating and/or ameliorating AHR;
in a subject in need thereof, comprising administering to the subject an effective amount of an α7nAChr agonist.

2. The method of claim 1, wherein the α7nAChr agonist comprises an active form of GTS-21, or an equivalent or a pharmaceutically acceptable salt thereof.

3. (canceled)

4. The method of claim 2, wherein the active form of GTS-21 comprises 4-OH-GTS-21 and an equivalent interacts with the α7-specific residues located at loop C (Arg182 and Glu185) and loop F (Glu158 and Asp160).

5. The method of claim 1, further comprising administering to the subject an effective amount of an additional agent selected from the group of: an anti-inflammatory agent, a steroid, a corticosteroid, an antibiotic, an alpha blocker, a beta blocker, an antihistamine, a blocking antibody against Th2 cytokines, an IgE, a LABA, a SABA, a LAMA, and an MABA, to the subject, that is optionally administered concurrently or sequentially in one or more administrations.

6.-7. (canceled)

8. A method for one or more of:
a. reducing ILC2 effector function;
b. repressing IC2-dependent AHR;
c. decreasing expression of ILC2 transcription factor GATA-3;
d. decreasing expression of ILC2 inflammatory modulator NF-κB;
e. reducing phosphorylation of kinase IKKα/β; or
f. reducing ILC2-mediated cytokine production;
comprising contacting an ILC2-cell expressing α7-nicotine acetylcholine receptor (α7nAChr) with an effective amount of an α7nAChr agonist, that is optionally administered in one or more doses or administrations.

9. The method of claim 8, wherein the α7nAChr agonist comprises an active form of GTS-21, or an equivalent or a pharmaceutically acceptable salt thereof.

10. (canceled)

11. The method of claim 8, wherein the active form of GTS-21 comprises 4-OH-GTS-21 and an equivalent interacts with the α7-specific residues located at loop C (Arg182 and Glu185) and loop F (Glu158 and Asp160).

12. The method of claim 8, further comprising contacting the cell with an effective amount of an additional agent selected from the group of: an anti-inflammatory agent, a steroid, a corticosteroid, an antibiotic, an alpha blocker, a beta blocker, an antihistamine, a blocking antibody against Th2 cytokines, an IgE, a LABA, a SABA, a LAMA, and an MABA, to the subject, that is optionally contacted concurrently or sequentially in one or more administrations.

13.-15. (canceled)

16. The method for treating an inflammatory condition related to ILC2 effector function in a subject in need thereof, comprising administering to the subject an effective amount of an α7nAChr agonist.

17. The method of claim 16, wherein the inflammatory condition is selected from the group of: acute and chronic inflammatory disorders, rheumatoid arthritis, COPD, irritable bowel syndrome, Crohn's disease, ulcerative colitis (UC), a food allergy, allergic and non-allergic asthma, non-allergic asthma, lung inflammation, mucosal inflammation, nasal polyps, eczema, and atopic dermatitis.

18. The method of claim 16, wherein the α7nAChr agonist comprises an active form of GTS-21, or an equivalent or a pharmaceutically acceptable salt thereof.

19. (canceled)

20. The method of claim 18, wherein the active form of GTS-21 comprises 4-OH-GTS-21 and the equivalent interacts with the α7-specific residues located at loop C (Arg182 and Glu185) and loop F (Glu158 and Asp160).

21. The method of claim 16, further comprising administering to the subject an effective amount of an additional agent selected from the group of: an anti-inflammatory agent, a steroid, a corticosteroid, an antibiotic, an alpha blocker, a beta blocker, an antihistamine, a blocking antibody against Th2 cytokines, an IgE, a LABA, a SABA, a LAMA, and an MABA, to the subject, that is optionally administered concurrently or sequentially in one or more administrations.

22.-23. (canceled)

24. A method for screening for an α7nAChr agonist, comprising contacting an ILC2 cell expressing α7nAChr or tissue sample comprising ILC2 cells expressing α7nAChr, with a test agent and evaluating for the cell or tissue for one or more of:
a. reducing ILC2 effector function
b. decreasing expression of ILC2 transcription factor GATA-3;
c. decreasing expression of TLC2 inflammatory modulator NF-κB;
d. reducing phosphorylation of kinase IKKα/β;
e. reducing ILC2-mediated cytokine production;
f. treating and/or ameliorating AHR;
g. treating and/or ameliorating allergic inflammation; or
h. treating and/or ameliorating an inflammatory condition or response related to ILC-2 effector function,
wherein a measured response of a. to h. identifies the test agent as a possible α7nAChr agonist.

25. The method of claim 24, further comprising comparing the activity of the test agent with the α7nAChr agonist activity of 4-OH-GTS-21.

26.-29. (canceled)

30. A method for screening for an agent that treats or ameliorates ILC2-mediated AHR and/or allergic inflammation, or an inflammatory disease or condition related to ILC2 effector function, comprising administering to a subject suffering ILC2-mediated AHR and/or allergic inflammation or the inflammatory disease or condition an amount of ILC2-mediated AHR and/or allergic inflammation, wherein a measured response identifies the test agent as a possible α7nAChr agonist.

31.-33. (canceled)


SEQUENCE LISTING (4 sequences)

SEQ ID NO: 1 - DNA, 22 bp, Artificial Sequence
  Description of Artificial Sequence Synthetic primer
ctctgactgtcttcatgctgct

SEQ ID NO: 2 - DNA, 18 bp, Artificial Sequence
  Description of Artificial Sequence Synthetic primer
atcatggtgctggcgaag

SEQ ID NO: 3 - DNA, 20 bp, Artificial Sequence
  Description of Artificial Sequence Synthetic primer
cgtccagtacattgcagacc

SEQ ID NO: 4 - DNA, 20 bp, Artificial Sequence
  Description of Artificial Sequence Synthetic primer
atgaccatggccacgtattt